SEQUENCE LISTING (65 sequences)

SEQ ID NO: 1 - DNA, 64 bp, synthetic construct
  source 1..64: mol_type = other DNA
tgtggagggattgaaggatatccacccacgcgttactcaccgtatgtggagggattgaaggata

SEQ ID NO: 2 - DNA, 68 bp, synthetic construct
  source 1..68: mol_type = other DNA
tgtggagggattgaaggatacgacgccgccttttaaacttctttctactgtggagggattgaaggata

SEQ ID NO: 3 - DNA, 68 bp, synthetic construct
  source 1..68: mol_type = other DNA
tgtggagggattgaaggataactgtttctgatgcagttcttcggtacttgtggagggattgaaggata

SEQ ID NO: 4 - DNA, 69 bp, synthetic construct
  source 1..69: mol_type = other DNA
tgtggagggattgaaggatacggagcgtcagttacagaccagagagggctgtggagggattgaaggata

SEQ ID NO: 5 - DNA, 66 bp, synthetic construct
  source 1..66: mol_type = other DNA
tgtggagggattgaaggatagtgcctgtctctttgtccccgaaccctgtggagggattgaaggata

SEQ ID NO: 6 - DNA, 67 bp, synthetic construct
  source 1..67: mol_type = other DNA
tgtggagggattgaaggatatcgtaagagatccgcttgccttcagtctgtggagggattgaaggata

SEQ ID NO: 7 - DNA, 67 bp, synthetic construct
  source 1..67: mol_type = other DNA
tgtggagggattgaaggataccgctggctccaaaggttacctcaggctgtggagggattgaaggata

SEQ ID NO: 8 - DNA, 68 bp, synthetic construct
  source 1..68: mol_type = other DNA
tgtggagggattgaaggataaaatgcgaaagtagcttttatccggatatgtggagggattgaaggata

SEQ ID NO: 9 - DNA, 69 bp, synthetic construct
  source 1..69: mol_type = other DNA
ataggaaatggtggtagtgttggagttcgttcgacttgcatgtatttccataggaaatggtggtagtgt

SEQ ID NO: 10 - DNA, 68 bp, synthetic construct
  source 1..68: mol_type = other DNA
ataggaaatggtggtagtgtggcaccccaccatctagctaatacgggcataggaaatggtggtagtgt

SEQ ID NO: 11 - DNA, 68 bp, synthetic construct
  source 1..68: mol_type = other DNA
ataggaaatggtggtagtgtattacagagttttacgagccgaaacggaataggaaatggtggtagtgt

SEQ ID NO: 12 - DNA, 67 bp, synthetic construct
  source 1..67: mol_type = other DNA
ataggaaatggtggtagtgtaagagtttccgatgcacttcttcgcaaataggaaatggtggtagtgt

SEQ ID NO: 13 - DNA, 69 bp, synthetic construct
  source 1..69: mol_type = other DNA
ataggaaatggtggtagtgttttctccaacacttagcattcatcgtaatataggaaatggtggtagtgt

SEQ ID NO: 14 - DNA, 68 bp, synthetic construct
  source 1..68: mol_type = other DNA
ataggaaatggtggtagtgtacaccgaagggaacgtctaatctctatcataggaaatggtggtagtgt

SEQ ID NO: 15 - DNA, 69 bp, synthetic construct
  source 1..69: mol_type = other DNA
ataggaaatggtggtagtgtaatgctggcaactgataataagggttcgcataggaaatggtggtagtgt

SEQ ID NO: 16 - DNA, 68 bp, synthetic construct
  source 1..68: mol_type = other DNA
ataggaaatggtggtagtgtcaaagcctacaatccgaactgagaaaccataggaaatggtggtagtgt

SEQ ID NO: 17 - DNA, 69 bp, synthetic construct
  source 1..69: mol_type = other DNA
agagtgagtagtagtggagtgtagtttccaaatgttatcccccactattagagtgagtagtagtggagt

SEQ ID NO: 18 - DNA, 66 bp, synthetic construct
  source 1..66: mol_type = other DNA
agagtgagtagtagtggagtcagaatgtggccgatcaacctctgtcagagtgagtagtagtggagt

SEQ ID NO: 19 - DNA, 68 bp, synthetic construct
  source 1..68: mol_type = other DNA
agagtgagtagtagtggagtagaaacaacagagttttacgacccgtttagagtgagtagtagtggagt

SEQ ID NO: 20 - DNA, 67 bp, synthetic construct
  source 1..67: mol_type = other DNA
agagtgagtagtagtggagtaaacagtttccgatgcacttcctcccaagagtgagtagtagtggagt

SEQ ID NO: 21 - DNA, 68 bp, synthetic construct
  source 1..68: mol_type = other DNA
agagtgagtagtagtggagttttctccaacacctagcattcatcgaaaagagtgagtagtagtggagt

SEQ ID NO: 22 - DNA, 65 bp, synthetic construct
  source 1..65: mol_type = other DNA
agagtgagtagtagtggagttgcaaccatgcaccacctgtcaaaaagagtgagtagtagtggagt

SEQ ID NO: 23 - DNA, 67 bp, synthetic construct
  source 1..67: mol_type = other DNA
agagtgagtagtagtggagtgcctgtcaccggcagtcttactagtcaagagtgagtagtagtggagt

SEQ ID NO: 24 - DNA, 65 bp, synthetic construct
  source 1..65: mol_type = other DNA
agagtgagtagtagtggagtgtagtcccaccttagacggctccgaagagtgagtagtagtggagt

SEQ ID NO: 25 - DNA, 66 bp, synthetic construct
  source 1..66: mol_type = other DNA
ttggaggtgtagggagtaaaccagatcccgttcgacttgcatgacattggaggtgtagggagtaaa

SEQ ID NO: 26 - DNA, 68 bp, synthetic construct
  source 1..68: mol_type = other DNA
ttggaggtgtagggagtaaagtacatgccactaaacactttcccatgtttggaggtgtagggagtaaa

SEQ ID NO: 27 - DNA, 68 bp, synthetic construct
  source 1..68: mol_type = other DNA
ttggaggtgtagggagtaaatcggcgcttattcgaaaggtacactgtgttggaggtgtagggagtaaa

SEQ ID NO: 28 - DNA, 66 bp, synthetic construct
  source 1..66: mol_type = other DNA
ttggaggtgtagggagtaaataaagtctcccctactgcactcttcgttggaggtgtagggagtaaa

SEQ ID NO: 29 - DNA, 68 bp, synthetic construct
  source 1..68: mol_type = other DNA
ttggaggtgtagggagtaaagtctatctaatcctgttcgctcccctgcttggaggtgtagggagtaaa

SEQ ID NO: 30 - DNA, 65 bp, synthetic construct
  source 1..65: mol_type = other DNA
ttggaggtgtagggagtaaagacaacctgccccgaaaggaaaggtttggaggtgtagggagtaaa

SEQ ID NO: 31 - DNA, 67 bp, synthetic construct
  source 1..67: mol_type = other DNA
ttggaggtgtagggagtaaaagcttgcagactacgatccgaactctcttggaggtgtagggagtaaa

SEQ ID NO: 32 - DNA, 67 bp, synthetic construct
  source 1..67: mol_type = other DNA
ttggaggtgtagggagtaaaaggatcacgagcctcaccttagacccgttggaggtgtagggagtaaa

SEQ ID NO: 33 - DNA, 69 bp, synthetic construct
  source 1..69: mol_type = other DNA
agggtgtgtttgtaaagggtgttaccaaattcatctaggcaagctccgaagggtgtgtttgtaaagggt

SEQ ID NO: 34 - DNA, 69 bp, synthetic construct
  source 1..69: mol_type = other DNA
agggtgtgtttgtaaagggtcgttccatccaagagtgatagcagaaggtagggtgtgtttgtaaagggt

SEQ ID NO: 35 - DNA, 69 bp, synthetic construct
  source 1..69: mol_type = other DNA
agggtgtgtttgtaaagggtaagcactaccaacagagctttacgagggcagggtgtgtttgtaaagggt

SEQ ID NO: 36 - DNA, 69 bp, synthetic construct
  source 1..69: mol_type = other DNA
agggtgtgtttgtaaagggtagatgcactcaagttcaacagtttctctaagggtgtgtttgtaaagggt

SEQ ID NO: 37 - DNA, 64 bp, synthetic construct
  source 1..64: mol_type = other DNA
agggtgtgtttgtaaagggttacccacctgtctcagcgtccggcagggtgtgtttgtaaagggt

SEQ ID NO: 38 - DNA, 66 bp, synthetic construct
  source 1..66: mol_type = other DNA
agggtgtgtttgtaaagggttgctttcagagatccgcttgcctagcagggtgtgtttgtaaagggt

SEQ ID NO: 39 - DNA, 65 bp, synthetic construct
  source 1..65: mol_type = other DNA
agggtgtgtttgtaaagggttagaccggctttgggtgttacactgagggtgtgtttgtaaagggt

SEQ ID NO: 40 - DNA, 69 bp, synthetic construct
  source 1..69: mol_type = other DNA
agggtgtgtttgtaaagggtgcttcaccctaatcatctgtcctaccaatagggtgtgtttgtaaagggt

SEQ ID NO: 41 - DNA, 64 bp, synthetic construct
  source 1..64: mol_type = other DNA
aggttaggttgagaataggaagatggttcagctcgctcgacaacaggttaggttgagaatagga

SEQ ID NO: 42 - DNA, 68 bp, synthetic construct
  source 1..68: mol_type = other DNA
aggttaggttgagaataggagaagccgccttttataagctgatcaacgaggttaggttgagaatagga

SEQ ID NO: 43 - DNA, 69 bp, synthetic construct
  source 1..69: mol_type = other DNA
aggttaggttgagaataggacattctacggattgcactagatgtcatctaggttaggttgagaatagga

SEQ ID NO: 44 - DNA, 66 bp, synthetic construct
  source 1..66: mol_type = other DNA
aggttaggttgagaataggaccggctcctcgttgtactgtccaaacaggttaggttgagaatagga

SEQ ID NO: 45 - DNA, 68 bp, synthetic construct
  source 1..68: mol_type = other DNA
aggttaggttgagaataggagtaccgaagtgaatctgttggttcacgaaggttaggttgagaatagga

SEQ ID NO: 46 - DNA, 68 bp, synthetic construct
  source 1..68: mol_type = other DNA
aggttaggttgagaataggactgcttacgccgccttttataagctctaaggttaggttgagaatagga

SEQ ID NO: 47 - DNA, 69 bp, synthetic construct
  source 1..69: mol_type = other DNA
aggttaggttgagaataggaagaggattgcactagatgtcaagaccaccaggttaggttgagaatagga

SEQ ID NO: 48 - DNA, 66 bp, synthetic construct
  source 1..66: mol_type = other DNA
aggttaggttgagaataggatcctgctcctcgttgtactgtcctaaaggttaggttgagaatagga

SEQ ID NO: 49 - DNA, 69 bp, synthetic construct
  source 1..69: mol_type = other DNA
gatgatgtagtagtaagggtgcggctttcctaacgtcattaccgaacatgatgatgtagtagtaagggt

SEQ ID NO: 50 - DNA, 69 bp, synthetic construct
  source 1..69: mol_type = other DNA
gatgatgtagtagtaagggtaccagcctttaccttaccaactagctttagatgatgtagtagtaagggt

SEQ ID NO: 51 - DNA, 68 bp, synthetic construct
  source 1..68: mol_type = other DNA
gatgatgtagtagtaagggtatgatggcactagatgtcaagacctccagatgatgtagtagtaagggt

SEQ ID NO: 52 - DNA, 68 bp, synthetic construct
  source 1..68: mol_type = other DNA
gatgatgtagtagtaagggtttgagaacagctttcagagattcgcaacgatgatgtagtagtaagggt

SEQ ID NO: 53 - DNA, 66 bp, synthetic construct
  source 1..66: mol_type = other DNA
gatgatgtagtagtaagggttcaatctgttagttccgctcgctgctgatgatgtagtagtaagggt

SEQ ID NO: 54 - DNA, 69 bp, synthetic construct
  source 1..69: mol_type = other DNA
gatgatgtagtagtaagggtaacaagcctttaccttaccaactagcattgatgatgtagtagtaagggt

SEQ ID NO: 55 - DNA, 65 bp, synthetic construct
  source 1..65: mol_type = other DNA
gatgatgtagtagtaagggtacgcacctgtcttagcgtcccccttgatgatgtagtagtaagggt

SEQ ID NO: 56 - DNA, 68 bp, synthetic construct
  source 1..68: mol_type = other DNA
gatgatgtagtagtaagggttgagaacagctttcagagattcgctacggatgatgtagtagtaagggt

SEQ ID NO: 57 - DNA, 38 bp, synthetic construct
  source 1..38: mol_type = other DNA
tagagttgatagagggagaagctgcctcccgtaggagt

SEQ ID NO: 58 - DNA, 20 bp, synthetic construct
  source 1..20: mol_type = other DNA
  misc_feature 1: note = Alexa Fluor 488 (NHS Ester)
tatccttcaatccctccaca

SEQ ID NO: 59 - DNA, 20 bp, synthetic construct
  source 1..20: mol_type = other DNA
  misc_feature 1: note = Alexa Fluor 546 (NHS Ester)
acactaccaccatttcctat

SEQ ID NO: 60 - DNA, 20 bp, synthetic construct
  source 1..20: mol_type = other DNA
  misc_feature 1: note = 6-ROX (NHS Ester)
  misc_feature 20: note = ROX (NHS Ester)
actccactactactcactct

SEQ ID NO: 61 - DNA, 20 bp, synthetic construct
  source 1..20: mol_type = other DNA
  misc_feature 1: note = ATTO 610 (NHS Ester)
tttactccctacacctccaa

SEQ ID NO: 62 - DNA, 20 bp, synthetic construct
  source 1..20: mol_type = other DNA
  misc_feature 1: note = Alexa Fluor 647 (NHS Ester)
accctttacaaacacaccct

SEQ ID NO: 63 - DNA, 20 bp, synthetic construct
  source 1..20: mol_type = other DNA
  misc_feature 1: note = DyLight 510-LS (NHS Ester)
  misc_feature 20: note = DyLight-510-LS (NHS Ester)
tcctattctcaacctaacct

SEQ ID NO: 64 - DNA, 20 bp, synthetic construct
  source 1..20: mol_type = other DNA
  misc_feature 1: note = Alexa Fluor 405 (NHS Ester)
ttctccctctatcaactcta

SEQ ID NO: 65 - DNA, 20 bp, synthetic construct
  source 1..20: mol_type = other DNA
  misc_feature 1: note = Alexa Fluor 532 (NHS Ester)
  misc_feature 20: note = Alexa Fluor 532 (NHS Ester)
acccttactactacatcatc

(19) United States
(12) Patent Application Publication
(10) Pub. No.: US 2026/0286447 A1
(43) Pub. Date: Sep. 24, 2026

(54) SCREENING FOR HOST-MICROBE INTERACTIONS

(71) Applicant: Kanvas Biosciences, Inc., Monmouth Junction, NJ (US)

(72) Inventors: Philip S. Burnham, Monmouth Junction, NJ (US); Matthew Cheng, Monmouth Junction, NJ (US); Hao Shi, Monmouth Junction, NJ (US); Prateek Sehgal, Monmouth Junction, NJ (US); Gaetano Scuderi, Monmouth Junction, NJ (US)

(73) Assignee: Kanvas Biosciences, Inc., Monmouth Junction, NJ (US)

(21) Appl. No.: 19/471,569

(22) PCT Filed: Apr. 1, 2024
(86) PCT No.: PCT/US2024/022443

§ 371 Date: Oct. 1, 2025

Related U.S. Application Data
(60) Provisional application No. 63/493,817, filed on Apr. 3, 2023.

(51) Int. Cl. C12Q 1/689; C12Q 1/02; C12Q 1/6841; C40B 40/06; G01N 21/64

(52) U.S. Cl. CPC C12Q 1/689; C12Q 1/025; C12Q 1/6841; C40B 40/06; G01N 21/6458; G01N 21/6486; C12Q 2600/136

(57) ABSTRACT

Described herein are methods for screening potential live biotherapeutic products. In some embodiments, the method includes (i) providing a sample, a pathogen, at least one microbial strain, and at least one set of probes; (ii) performing an imaging assay; and (iii) quantifying the association between the at least one microbial strain and the sample as compared to the association between the pathogen and the sample, thereby determining the therapeutic potential of the at least one microbial strain to treat or prevent a disease caused by the pathogen.

### CROSS-REFERENCE TO RELATED APPLICATIONS

This application claims the benefit of and priority to U.S. Provisional Patent Application No. 63/493,817, filed Apr. 3, 2023, the disclosure of which is hereby incorporated by reference in its entirety for all purposes.

### SEQUENCE LISTING

This application incorporates by reference in its entirety the Sequence Listing entitled “Screening for Host-Microbe Interactions.xml” (65,213 bytes), which was created on Mar. 29, 2024, and filed electronically herewith.

### TECHNICAL FIELD

This invention relates to methods for screening potential live biotherapeutic products.

### BACKGROUND

The microbiome plays a crucial role in the metabolism and efficacy of drugs and for diseases that can occur both within and outside the gastrointestinal (GI) tract. A new class of therapeutics, live biotherapeutic products (LBPs), aims to directly manipulate the microbiome by administering live microorganisms to prevent disease or improve treatment response. While several dozen LBP candidates are under clinical trials, their success rate is low, with only a single LBP recently passing the FDA special committee approval process.

High-throughput screening has become an important component in drug discovery, allowing rapid evaluation of numerous therapeutic compounds. However, no such process exists for live biotherapeutic product (LBP) development, which has hindered progress in meeting FDA requirements that include proof of competition, safety, and therapeutic effect. Accordingly, there is an acute need for new technology to enable LBP discovery.

### SUMMARY

In one aspect, a screening method can include:
(i) providing:
   (a) a sample;
   (b) a pathogen;
   (c) at least one microbial strain, and
   (d) at least one set of probes, wherein the at least one set of probes comprises at least one encoding probe and at least one emissive readout probe;
(ii) performing an imaging assay, wherein the imagining assay comprises:
   (A) contacting the (a) sample with the (b) pathogen and the (c) at least one microbial strain to produce a first complex;
   (B) contacting the at least one encoding probe with the first complex to form a second complex, wherein the at least one encoding probe comprises a targeting sequence and a landing pad sequence,
   (C) contacting the at least one emissive readout probe with the second complex, wherein the at least one emissive readout probe comprises a label and a sequence complementary to the landing pad sequence;
   (D) acquiring one or more emission spectra from the at least one emissive readout probe;
   (E) repeating the aforementioned steps for at least one different encoding probe;
   (F.1) determining the spectra of “signal” and assigning them to a species of the at least one microbial strain;
   (F.2) determining the spectra of “signal” and assigning them to a species of the pathogen;
   (G) decoding the spectra into a single, targeted transcript through means of signal deconvolution, error correction, comparison to reference standards; and
(iii) quantifying the association between the at least one microbial strain and the sample as compared to the association between the pathogen and the sample, thereby determining the therapeutic potential of the at least one microbial strain to treat or prevent a disease caused by the pathogen.

In another aspect, a screening method can include:
(i) providing:
   (a) a sample;
   (b) a pathogen;
   (c) at least one microbial strain, and
   (d) at least one set of probes, wherein the at least one set of probes comprises at least one encoding probe and at least one emissive readout probe;
(ii) performing an imaging assay, wherein the imagining assay comprises:
   (A) contacting the (a) sample with the (b) pathogen and the (c) at least one microbial strain to produce a first complex;
   (B) contacting the at least one encoding probe with the first complex to form a second complex, wherein the at least one encoding probe comprises a targeting sequence and a landing pad sequence,
   (C) contacting the at least one emissive readout probe with the second complex, wherein the at least one emissive readout probe comprises a label and a sequence complementary to the landing pad sequence;
   (D) acquiring one or more emission spectra from the at least one emissive readout probe;
   (E) repeating the aforementioned steps for at least one different encoding probe;
   (F.1) determining the spectra of “signal” and assigning them to a species of the at least one microbial strain;
   (F.2) determining the spectra of “signal” and assigning them to a species of the pathogen;
   (G) decoding the spectra into a single, targeted transcript through means of signal deconvolution, error correction, comparison to reference standards; and
(iii) quantifying the association between the at least one microbial strain and the sample as compared to the association between the pathogen and the sample, thereby determining the potential of the at least one microbial strain to be a live biotherapeutic product (LBP).

In some embodiments, the sample comprises at least one cell, a cell suspension, a tissue biopsy, a tissue specimen, bone biopsies, organoids, three-dimensional hydrogel scaffolds, transwell systems, or plant biopsies. In some embodiments, the sample is a cell; optionally, the cell is a bacterial or eukaryotic cell.

In some embodiments, the pathogen is a disease-causing microorganism selected from the group consisting of a bacterium, a fungus, a virus, an archaea, and a parasite, or a fragment thereof. In some embodiments, the pathogen is a disease-causing microorganism that causes a urogenital, skin, lung, or gastrointestinal disease.

In some embodiments, the at least one microbial strain is a strain derived from a microbe selected from the group consisting of a bacterium, a fungus, a virus, an archaea, and a parasite. In some embodiments, the at least one microbial strain is a bacterial strain. In some embodiments, the at least one microbial strain is a genetically engineered strain. In some embodiments, the at least one microbial strain is a genetically engineered bacterial strain.

In some embodiments, the targeting sequence targets at least one of messenger RNA (mRNA), micro RNA (miRNA), long non-coding RNA (lncRNA), ribosomal RNA (rRNA), small interfering RNA (siRNA), transfer RNA (tRNA), Crispr RNA (crRNA), trans-activating cirspr RNA (tracrRNA), mitochondria RNA, Intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigenic target.

In some embodiments, the targeting sequence targets a 16S rRNA sequence, a 5S rRNA sequence, and/or a 23S rRNA sequence in the microbial strain. In some embodiments, the targeting sequence targets a 16S rRNA sequence, a 5S rRNA sequence, and/or a 23S rRNA sequence in the pathogen.

In some embodiments, the targeting sequence targets a 18S rRNA sequence, a 5.8S rRNA sequence, and/or a 28S rRNA sequence in the microbial strain, wherein the microbial strain is a eukaryotic microbial strain.

In some embodiments, the targeting sequence targets transcripts that confer taxonomic specificity.

Another aspect of the present disclosure provides a library of constructs, each construct comprising:
(a) a target cell;
(b) a first plurality of encoding and readout probes, wherein each encoding probe comprises a targeting sequence that is a region of interest on a nucleotide of a pathogen and a landing pad sequence; and each readout probe comprises a label and a sequence complementary to the landing pad sequence; and
(c) a second plurality of encoding and readout probes, wherein each encoding probe comprises a targeting sequence that is a region of interest on a nucleotide of a microbial strain and a landing pad sequence; and each readout probe comprises a label and a sequence complementary to the landing pad sequence.

Another aspect of the present disclosure provides a library of constructs, each construct comprising:
(a) a target cell;
(b) a first plurality of encoding and readout probes, wherein each encoding probe comprises a targeting sequence that is a region of interest on a nucleotide of a pathogen and a landing pad sequence; and each readout probe comprises a label and a sequence complementary to the landing pad sequence; and
(c) a second plurality of encoding and readout probes, wherein each encoding probe comprises a targeting sequence that is a region of interest on a nucleotide of a bacterial strain and a landing pad sequence; and each readout probe comprises a label and a sequence complementary to the landing pad sequence.

In another aspect, the present disclosure provides methods for determining the spatial location of a complex, comprising:
(i) providing:
   (a) a target cell;
   (b) a pathogen;
   (c) at least one microbial strain, and
   (d) at least one set of probes, wherein the at least one set of probes comprises at least one encoding probe and at least one emissive readout probe;
(ii) performing an imaging assay, wherein the imagining assay comprises:
   (A) contacting the (a) target cell with the (b) pathogen and the (c) at least one microbial strain to produce a first complex;
   (B) contacting the at least one encoding probe with the first complex to form a second complex, wherein the at least one encoding probe comprises a targeting sequence and a landing pad sequence,
   (C) contacting the at least one emissive readout probe with the second complex, wherein the at least one emissive readout probe comprises a label and a sequence complementary to the landing pad sequence;
   (D) acquiring one or more emission spectra from the at least one emissive readout probe;
   (E) repeating the aforementioned steps for at least one different encoding probe;
   (F.1) determining the spectra of “signal” and assigning them to a species of the at least one microbial strain;
   (F.2) determining the spectra of “signal” and assigning them to a species of the pathogen;
   (G) decoding the spectra into a single, targeted transcript through means of signal deconvolution, error correction, comparison to reference standards;
   (H) performing a second assay to image the target cell; and
(iii) quantify the abundance of the at least one microbial strain to the target cell and/or the association between the pathogen to the target cell, thereby determining the spatial location of the complex,
wherein the complex is a target cell-microbial strain complex or a target cell-pathogen complex.

In another aspect, the present disclosure provides methods for determining the spatial location of a complex, comprising:
(i) providing:
   (a) a target cell;
   (b) a pathogen;
   (c) at least one bacterial strain, and
   (d) at least one set of probes, wherein the at least one set of probes comprises at least one encoding probe and at least one emissive readout probe;
(ii) performing an imaging assay, wherein the imagining assay comprises:
   (A) contacting the (a) target cell with the (b) pathogen and the (c) at least one bacterial strain to produce a first complex;
   (B) contacting the at least one encoding probe with the first complex to form a second complex, wherein the at least one encoding probe comprises a targeting sequence and a landing pad sequence,
   (C) contacting the at least one emissive readout probe with the second complex, wherein the at least one emissive readout probe comprises a label and a sequence complementary to the landing pad sequence;
   (D) acquiring one or more emission spectra from the at least one emissive readout probe;
   (E) repeating the aforementioned steps for at least one different encoding probe;
   (F.1) determining the spectra of “signal” and assigning them to a species of the at least one bacterial strain;
   (F.2) determining the spectra of “signal” and assigning them to a species of the pathogen;
   (G) decoding the spectra into a single, targeted transcript through means of signal deconvolution, error correction, comparison to reference standards;
   (H) performing a second assay to image the target cell; and
(iii) quantify the abundance of the at least one bacterial strain to the target cell and/or the association between the pathogen to the target cell, thereby determining the spatial location of the complex,
wherein the complex is a target cell-bacterial strain complex or a target cell-pathogen complex.

Other aspects, embodiments, and features as disclosed herein will be apparent from the following description, the drawings, and the claims.

### BRIEF DESCRIPTION OF THE DRAWINGS

FIG. 1 is a schematic drawing showing the potential measurement of interactions between various microbes (patterns). As shown in the schematic, proximity networks can be drawn between various microbes in measured images. An interaction matrix can then be constructed for different taxa (high numbers of interactions are shown with more stars (*)). To assess the significance of the measured proximities, and determine if certain taxon-taxon interactions are enriched or suppressed, the identities of each microbe can be randomly assigned while the positions and proximity network remain identical. A new interaction matrix can be computed and compared relative to the measured one.

FIG. 2 is a schematic drawing showing the setup of the microbial adhesion assay. (1) Host cells are seeded onto a substrate with media; (2) host cells grow/spread and form a stable monolayer. Therapeutic and pathogenic microbes can then be added to the cell monolayers (3) simultaneously (competition), (4) with the pathogenic microbe/microbiota added before the LBP or therapeutic microbe/microbiota (interference (A)), (5) with the LBP or therapeutic microbe/microbiota added before the pathogenic microbe/microbiota. In each modality, after some passage of time, all cells are fixed and the imaging assay is performed to map microbiota.

FIG. 3 is a schematic drawing showing the measurement output of the imaging assay. Collected images show microbes and cultured host cells. The host cell can be analyzed by performing cell segmentation and determining the morphometric properties of the host cells (e.g., cell area, nuclear eccentricity, etc.). Gene expression measurements can also be performed. In another aspect, our computational platform allows for microbial cells to be segmented, measured, and taxonomically classified. For each host cell, we can generate a profile that includes the number of each microbial taxa in direct proximity, in addition to the host cell characteristics.

FIG. 4 shows that gene expression measurements in vaginal epithelial cells show distinct expression changes with adherence of G. vaginalis. On the left is a volcano plot showing the log 2 fold change between the G. vaginalis adhered cells and the control (no microbe) cells), while mean expression shows the average gene count. Triangles show significant (p<0.05) enriched or suppressed genes. On the right is a gene ontological analysis that was performed for genes suppressed (negative values) or enriched (positive values) in the presence of G. vaginalis.

FIGS. 5A-5C show that the screening platform disclosed herein allows us to evaluate adherence of pathogenic bacteria in absence of any live biotherapeutic product strains (FIG. 5A) and in the presence of any or all LBP strains (FIG. 5B) [in both cases, G. vaginalis is shown in green while Lactobacillus species are shown in other colors]. For each condition, we determined the fraction of cells where at least one G. vaginalis cell is adherent (y-axis). For each condition, the mean fraction of G. vaginalis adherent VECS is shown in FIG. 5C. Significance was determined using a 1-way ANOVA followed by a post-hoc comparison of means (Tukey).

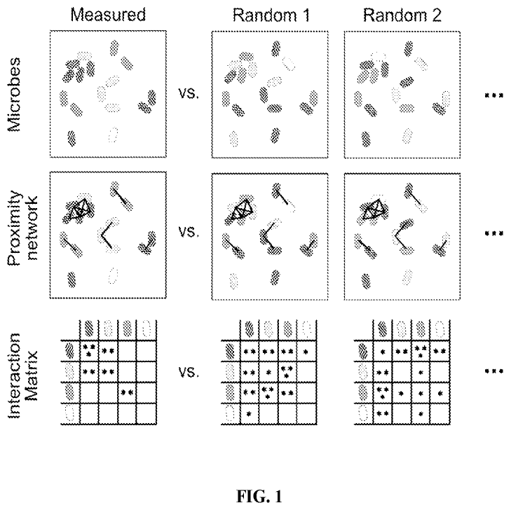

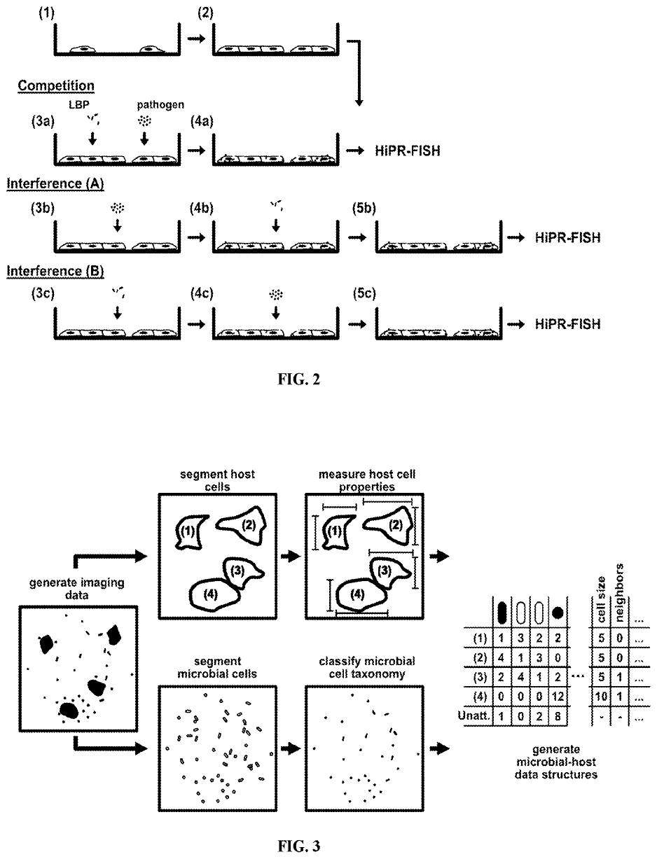

### INCORPORATION BY REFERENCE

All publications, patents, and patent applications mentioned in this specification are herein incorporated by reference to the same extent as if each individual publication, patent, or patent application was specifically and individually indicated to be incorporated by reference.

In particular, the entire contents of each of the following patent applications are incorporated herein by reference in their entireties: PCT Application Nos.: PCT/US2019/021088; PCT/US2022/080355; and PCT/US2023/062917; and U.S. application Ser. Nos. 16/978,891; 18/058,171; and Ser. No. 18/171,850.

### DETAILED DESCRIPTION

It is to be appreciated that certain aspects, modes, embodiments, variations, and features of the present methods and compositions are described below in various levels of detail in order to provide a substantial understanding of the present disclosure.

### Definitions

Where values are described as ranges, endpoints are included. Furthermore, it will be understood that such disclosure includes the disclosure of all possible sub-ranges within such ranges, as well as specific numerical values that fall within such ranges irrespective of whether a specific numerical value or specific sub-range is expressly stated.

“5′-end” and “3′-end” refers to the directionality, e.g., the end-to-end orientation of a nucleotide polymer (e.g., DNA). The 5′-end of a polynucleotide is the end of the polynucleotide that has the fifth carbon.

The term “about,” as used herein, refers to +/−10% of a recited value.

“Complementary” refers to the topological compatibility or matching together of interacting surfaces of two nucleotides as understood by those of skill in the art. Thus, two sequences are “complementary” to one another if they are capable of hybridizing to one another to form a stable anti-parallel, double-stranded nucleic acid structure. A first nucleotide is complementary to a second nucleotide if the nucleotide sequence of the first nucleotide is substantially identical to the nucleotide sequence of the nucleotide binding partner of the second nucleotide, or if the first nucleotide can hybridize to the second nucleotide under stringent hybridization conditions. Thus, the nucleotide whose sequence is 5′-TATAC-3′ is complementary to a nucleotide whose sequence is 5′-GTATA-3′.

“Nucleotides,” “Nucleic acids,” “polynucleotide” or “oligonucleotide” refer to a polymeric-form of DNA and/or RNA (e.g., ribonucleotides, deoxyribonucleotides, or analogs thereof) of any length; e.g., a sequence of two or more ribonucleotides or deoxyribonucleotides. As used herein, the term “nucleotides” includes double- and single-stranded DNA, as well as double- and single-stranded RNA; it also includes modified and unmodified forms of a nucleotide (modifications to and of a nucleotide, for example, can include methylation, phosphorylation, and/or capping). In some embodiments, a nucleotide can be one of the following: a gene or gene fragment; genomic DNA; genomic DNA fragment; exon; intron; messenger RNA (mRNA); transfer RNA (tRNA); ribosomal RNA (rRNA); ribozyme; cDNA; recombinant nucleotide; branched nucleotide; plasmid; vector; isolated DNA of any sequence; isolated RNA of any sequence; any DNA described herein, any RNA described herein, primer or amplified copy of any of the foregoing.

In some embodiments, nucleotides can have any three-dimensional structure and may perform any function, known or unknown. The structure of nucleotides can also be referenced to by their 5′- or 3′-end or terminus, which indicates the directionality of the nucleotide sequence. Adjacent nucleotides in a single-strand of nucleotides are typically joined by a phosphodiester bond between their 3′ and 5′ carbons. However, different internucleotide linkages could also be used, such as linkages that include a methylene, phosphoramidate linkages, etc. This means that the respective 5′ and 3′ carbons can be exposed at either end of the nucleotide sequence, which may be called the 5′ and 3′ ends or termini. The 5′ and 3′ ends can also be called the phosphoryl (PO₄) and hydroxyl (OH) ends, respectively, because of the chemical groups attached to those ends. The term “nucleotides” also refers to both double- and single-stranded molecules.

In some embodiments, nucleotides can include modified nucleotides, such as methylated nucleotides and nucleotide analogs (including nucleotides with non-natural bases, nucleotides with modified natural bases such as aza- or deaza-purines, etc.). If present, modifications to the nucleotide structure can be imparted before or after assembly of the nucleotide sequence.

In some embodiments, the sequence of nucleotides can be interrupted by non-nucleotide components. One or more ends of the nucleotides can be protected or otherwise modified to prevent that end from interacting in a particular way (e.g. forming a covalent bond) with other nucleotides.

In some embodiments, nucleotides can be composed of a specific sequence of four nucleotide bases: adenine (A); cytosine (C); guanine (G); and thymine (T). Uracil (U) can also be present, for example, as a natural replacement for thymine when the nucleotide is RNA. Uracil can also be used in DNA. Thus, the term “sequence” refers to the alphabetical representation of nucleotides or any nucleic acid molecule, including natural and non-natural bases.

When used in terms of length, for example 20 nt, “nt” refers to nucleotide(s).

As used herein a “taxon” refers to a group of one or more populations of an organism or organisms. In some embodiments, a “taxon” refers to a phylum, a class, an order, a family, a genus, a species, or a strain. In some embodiments, the disclosure includes providing a list of taxa of microorganisms. In some embodiments, the list of taxa of microorganisms is selected from a list of phyla, a list of classes, a list of orders, a list of families, a list of genera, or a list of species, of microorganisms.

In analysis of a sample, a species can be a target of interest. For example, a species can include a taxonomic species.

A “pathogen” refers to any agent capable of causing an infection or a disease in a host, i.e., a cell or subject. In some embodiments, a pathogen is a microorganism or microbial fragment. In some embodiments, the pathogen may comprise a whole (infectious) pathogen cell, or a part of the pathogen cell, such as, a cell wall component of a microorganism. In some embodiments, the pathogen comprises a pathogen fragment, a pathogen debris, a pathogen nucleic acid, a pathogen lipoprotein, a pathogen surface glycoprotein, a pathogen membrane component, or a component released from the pathogen. In some embodiments, the pathogen is, is derived from, or is isolated from bacteria, fungus, prokaryote, virus, phage, or a misfolded protein (e.g., a prion). In some embodiments, the pathogen is genetically modified.

A “microbial strain” refers to any strain derived from a microbe. A “microbial strain” may also be any strain that can potentially ameliorate a disease phenotype. In some embodiments, a microbial strain is any strain derived from a microbe and which does not cause a disease in a host, i.e., a cell or subject. In some embodiments, a microbial strain produces a therapeutic effect. In some embodiments, a microbial strain is a strain that acts against the pathogen. A microbial strain used in the methods described herein may derive from a healthy cell or subject and/or may contain healthy or desirable microbes. In some embodiments, a microbial strain is selected from the group consisting of a bacterium, a synthetic bacterium, a synthetic organism, a fungus, a virus, an archaea, a parasite, and a genetically modified organism. In some embodiments, a genetically modified organism is an organism wherein specific genes have been added, deleted, mutated, driven to high expressions, and/or suppressed to low expression levels. In some embodiments, the genetic modifications in the genetically modified organism are performed via electroporation, conjugation, transformation, transduction, CRISPR-Cas9 system, TALENs (Transcription Activator-Like Effector Nucleases), homologous recombination, site-specific recombinases, or bacterial artificial chromosomes. In some embodiments, a microbial strain is a bacterial strain such as a probiotic. The microbial strain of the methods described herein is to provide therapeutic/beneficial effects to the host and therefore does not cause a disease in said host. It is possible that microbial strains may be (or be derived from) strains that do not cause a disease in a normal host, but can cause disease in a subpopulation (e.g., an immunocompromised subject).

The “therapeutic potential” of a microbial (e.g., bacterial) strain refers to the therapeutic quality of the strain, i.e., the strain ability to provide a therapeutic benefit when administered to a subject. In some embodiments, the therapeutic potential of a microbial (e.g., bacterial) strain can be measured, quantified, determined, identified, or validated by its abundance near the (host) cells or cell walls and/or its ability to prevent adhesion of the pathogen to the (host) cells or cell walls. In some embodiments, the therapeutic potential of a microbial (e.g., bacterial) strain can be measured, quantified, determined, identified, or validated by the response of the (host) cells or cell walls. Therapeutic potential includes, but is not limited to, a microbial (e.g., bacterial) strain ability to prevent adhesion of the pathogen to the (host) cells or cell walls and/or its ability to treat or prevent the disease caused by the pathogen in a subject.

In the event of any term having an inconsistent definition between this application and a referenced document, the term is to be interpreted as defined herein.

### Live Biotherapeutic Product Screen

There is an acute need for new technology to enable live biotherapeutic product discovery. The microbiome plays a crucial role in the metabolism and efficacy of drugs for diseases that can occur both within and outside the gastrointestinal (GI) tract. A new class of therapeutics, live biotherapeutic products (LBPs), aims to directly manipulate the microbiome by administering live microorganisms to prevent disease or improve treatment response. While several dozen LBP candidates are under clinical trials, their success rate is low, with only a single LBP recently passing the FDA special committee approval process. At the same time, there are many difficult-to-treat diseases with known connections to microbes and the microbiome which could benefit greatly from LBP studies; one such disease is atopic dermatitis (AD).

Atopic dermatitis is a chronic skin condition affecting over 10 million US adults, marked by red, itchy skin, Staphylococcus aureus (S. aureus) colonization, and dysbiosis of the skin microbiota. The disease triggers an extensive innate immune response, exacerbating symptoms. LBP candidates targeting S. aureus and promoting balanced skin microbiota, are currently in clinical trials. Progress in LBP research could be accelerated with an innovative LBP screening platform, optimizing efficacy, safety, and specificity for AD treatment and other skin conditions associated with bacterial infections.

Bacterial vaginosis (BV) is a condition of the vagina that affects millions of Americans annually. In general, BV is caused by microbiome dysbiosis and can lead to extensive colonization and biofilm formation by pathogens, including Gardnerella vaginalis. Therapeutics for treating and preventing BV are of paramount importance as it can result in serious complications, including increased risk of sexually transmitted infections, pelvic inflammatory disease, and adverse pregnancy outcomes. Antibiotics are frequently prescribed, but can disrupt the natural flora in the healthy vagina and the gastrointestinal tract. Further, there is a risk of recrudescence upon cessation of antibiotic therapy. LBPs could serve to restore and support the healthy vaginal microbiome and prevent colonization of pathogenic G. vaginalis.

Clostridioides difficile infection (CDI) is a potentially severe and life-threatening bacterial infection of the colon caused by the toxin-producing bacterium Clostridioides difficile. The use of antibiotics can trigger CDI since it can disrupt the normal balance of the gut microbiota, allowing C. difficile to reproduce and produce toxins. There are hundreds of thousands of cases of CDI annually, and it has a tendency to be recurrent. Ferring Pharmaceuticals recently received special committee approval by the FDA for an LBP to treat CDI, showing the effectiveness of the approach. More complex or targeted LBPs could serve to further improve treatment efficacy or reduce the risk of future relapses than the approved drug.

Lack of advanced technology hinders rapid LBP screening and development. High-throughput screening has become an important component in drug discovery, allowing rapid evaluation of numerous therapeutic compounds. However, there are no substantive platforms for screening LBPs, which has hindered progress in meeting FDA requirements that include proof of composition, viability, safety, and therapeutic effect. The current best-in-practice, in vitro LBP screening platform is the adhesion assay, where LBP candidate taxa and pathogenic taxa are introduced simultaneously (i.e., “competition”) or sequentially (i.e., “interference), in order to examine the prevention of pathogen colonization by other therapeutic bacteria. While the principles of the assays are sound, the measurement techniques for identifying adherent microbiota are limited in target multiplexity and spatial resolution. For example, shotgun metagenomic sequencing, while sensitive, destroys spatial context. Sequencing measurements are also confounded by abundant host genetic material and microbiota that may adhere to the underlying substrate, but not host cells. While imaging-based assays can enable spatial measurements, the currently-available methods have only enabled 2 or 3 targets to be detected. For example, gram-staining of adherent cells is typically used to examine the potential therapeutic effect of LBPs. However, this method is only possible if the LBP candidates are all of one gram-type and the pathogen is of another. Fluorescence in situ hybridization (FISH), has been used in limited cases, but also has not achieved the multiplexity needed to examine complex LBP candidates at species-level resolution.

The current state-of-the-art technologies for profiling the microbiome have predominantly focused on phenotypic measurements (e.g., cell culture) or genotypic measurements (e.g., metagenomic sequencing). However, these approaches fail to preserve the native spatial context of microbial communities. When analyzing the effect of LBPs on generating an intended effect, it is important to observe which members of the LBP are binding, which cells they are binding to, and, if they are being used to deter the colonization of a pathogen, how much of the pathogen is present. Thus, phenotypic and genetic/sequencing measurements are inadequate. The gold standard technique of Gram-staining, though allowing for spatial measurement, is over a century old and can only profile two cell types, significantly narrowing the LBP design scope and the range of addressable diseases. Additionally, fluorescence in situ hybridization (FISH) for LBP screening has only managed to measure a limited number of taxa at the genus level.

Gram-staining can be used to visualize bacterial cells bound to cultured host cells and thorough washing. After staining, the specimens can be viewed on a microscope and adherent bacterial cells can be enumerated. However, the technology is limited in its ability to only profile two cell types, thereby limiting LBP consortia to be of a different gram-stain from the pathogen. This significantly narrows the design space for LBPs and the type of diseases that can be approached using this screening method. For example, such screening would not be able to distinguish S. aureus pathogenic bacteria from possible LBP taxa from the healthy human skin microbiome, like Cutibacterium acnes, as both are gram-positive. Previous studies using FISH to screen LBPs have similarly only been able to measure a few taxa.

Accordingly, there is an acute need for new technology to enable live biotherapeutic product discovery.

The LBP screening method described herein presents several key advantages over existing techniques, examples of which follow. (1) Utilizing an imaging-based approach, our method can capture species in their native spatial context and reveal microorganism adhesion to host cells. (2) It enables the simultaneous detection of up to 1023 unique species within a single field of view, facilitating the profiling of intricate LBPs. (3) It delivers results within hours, enabling rapid turnaround times and high throughput for sample processing. (4) Seamlessly integrating gene expression measurements, it also allows for host cell response assessments, providing valuable insights into safety and efficacy.

High Phylogenetic Resolution microbiome mapping by Fluorescence in situ Hybridization (HiPR-FISH), developed by the Applicant, is a versatile technology that uses binary encoding, spectral imaging, and machine learning based decoding to create micron-scale maps of the locations and identities of hundreds of microbial species in complex communities. See, for example, Shi, H. et al. “Highly multiplexed spatial mapping of microbial communities.” Nature vol. 588, 7839 (2020): 676-681, PCT Patent Publication WO 2019/173555, filed Mar. 7, 2019; PCT Patent Application No. PCT/US2022/080355, filed on Nov. 24, 2022, and U.S. application Ser. No. 18/058,171, filed on Nov. 24, 2022. The contents of the aforementioned disclosures are each incorporated herein by reference in their entireties.

HiPR-FISH has been able to distinguish between 1023 unique targets in a single experiment. It has been applied to numerous specimen types including mammalian tissue, food products, biofilms, and cultured epithelial cells with adherent bacteria. The present disclosure provides methods to incorporate this technology in screening for potential LBPs.

Accordingly, a screening method can include:
(i) providing:
   (a) a sample;
   (b) a pathogen;
   (c) at least one microbial strain, and
   (d) at least one set of probes, wherein the at least one set of probes comprises at least one encoding probe and at least one emissive readout probe;
(ii) performing an imaging assay, wherein the imagining assay comprises:
   (A) contacting the (a) sample with the (b) pathogen and the (c) at least one microbial strain to produce a first complex;
   (B) contacting the at least one encoding probe with the first complex to form a second complex, wherein the at least one encoding probe comprises a targeting sequence and a landing pad sequence,
   (C) contacting the at least one emissive readout probe with the second complex, wherein the at least one emissive readout probe comprises a label and a sequence complementary to the landing pad sequence;
   (D) acquiring one or more emission spectra from the at least one emissive readout probe;
   (E) repeating the aforementioned steps for at least one different encoding probe;
   (F.1) determining the spectra of “signal” and assigning them to a species of the at least one bacterial strain;
   (F.2) determining the spectra of “signal” and assigning them to a species of the pathogen;
   (G) decoding the spectra into a single, targeted transcript through means of signal deconvolution, error correction, comparison to reference standards; and
(iii) quantifying the association between the at least one microbial strain and the sample as compared to the association between the pathogen and the sample, thereby determining the therapeutic potential of the at least one microbial strain to treat or prevent a disease caused by the pathogen.

In other circumstances, a screening method can include:
(i) providing:
   (a) a sample;
   (b) a pathogen;
   (c) at least one microbial strain, and
   (d) at least one set of probes, wherein the at least one set of probes comprises at least one encoding probe and at least one emissive readout probe;
(ii) performing an imaging assay, wherein the imagining assay comprises:
   (A) contacting the (a) sample with the (b) pathogen and the (c) at least one microbial strain to produce a first complex;
   (B) contacting the at least one encoding probe with the first complex to form a second complex, wherein the at least one encoding probe comprises a targeting sequence and a landing pad sequence,
   (C) contacting the at least one emissive readout probe with the second complex, wherein the at least one emissive readout probe comprises a label and a sequence complementary to the landing pad sequence;
   (D) acquiring one or more emission spectra from the at least one emissive readout probe;
   (E) repeating the aforementioned steps for at least one different encoding probe;
   (F.1) determining the spectra of “signal” and assigning them to a species of the at least one bacterial strain;
   (F.2) determining the spectra of “signal” and assigning them to a species of the pathogen;
   (G) decoding the spectra into a single, targeted transcript through means of signal deconvolution, error correction, comparison to reference standards; and
(iii) quantifying the association between the at least one microbial strain and the sample as compared to the association between the pathogen and the sample, thereby determining the potential of the at least one microbial strain to be a live biotherapeutic product (LBP).

In some embodiments, more than one pathogen, more than one microbial strain, and more than one type of probe set (e.g., encoding probe and emissive readout probes) may be introduced to a sample. For example, there may be at least 2, at least 5, at least 10, at least 25, at least 50, at least 75, at least 100, at least 300, or at least 1,000 distinguishable microbial strains that are introduced to a sample. Further, there may be at least 2, at least 5, at least 10, at least 25, at least 50, at least 75, at least 100, at least 300, at least 1,000, at least 3,000, at least 10,000, or at least 30,000 distinguishable probe sets that are introduced to a sample. In some embodiments, the distinct probes and/or microbial strains are introduced simultaneously. In some embodiments, the distinct probes and/or microbial strains are introduced sequentially.

The method of the present disclosure can simultaneously detect multiple microbial strain species alongside a pathogen species, demonstrating the ability to uniquely identify each member of a complex LBP candidate. The method can exhibit the high accuracy of HiPR-FISH and is semi-automated, enabling us to rapidly provide data and interpretation in several days. This competitive turnaround time rivals that of other high-throughput, high-multiplexity methods such as 16S and metagenomic sequencing. The present method automates probe design, bacterial cell segmentation and classification, and spatial biological analyses.

The method of the present disclosure can adopt a low magnification modality, to image up to 10 times more host cells in a single field of view (FOV), reducing the number of FOVs needed for statistically significant trends from 4 or more to just 1. This approach can reduce imaging time by 75% or more. Further, mRNA detection capabilities can also be integrated into the screening method, thereby enabling gene expression analysis simultaneously with the ability to examine biological processes occurring in response to the microbial strain.

### Sample

In some embodiments, the sample comprises at least one cell, a cell suspension, a tissue biopsy, a tissue specimen, bone biopsies, organoids, three-dimensional hydrogel scaffolds, transwell systems, or plant biopsies.

In some embodiments, the sample is a cell. In some embodiments, the cell is a bacterial cell. In some embodiments, the cell is a eukaryotic cell. In some embodiments, the eukaryotic cell is a unicellular organism including protozoa, chromista, algae, or fungi. In some embodiments, the eukaryotic cell is part of a multicellular organism from chromista, plantae, fungi, or animalia. In some embodiments, the sample is a tissue composed of cells. In some embodiments, the cell contains foreign DNA/RNA from viruses, plasmids, and bacteria. In some embodiments, the cell has been genetically modified or synthetically engineered. In some embodiments, the cell has been genetically modified.

In some embodiments, the sample can include a plurality of cells. In some embodiments, each cell in the plurality of cells can include a specific targeting sequence, which may or may not be the same from the other targeting sequences.

In some embodiments, the sample is a whole organism.

In some embodiments, the sample comprises a plurality of cells. In some embodiments, the sample comprises a plurality of cells that contain the pathogen.

In some embodiments, the sample is obtained from a patient diagnosed with, or suspected to be suffering from an infection, disease, or disorder. In some embodiments, the patient has been diagnosed with, or is suspected to be suffering from a bacterial, viral, fungal, or parasitic infection. In some embodiments, the infection includes, but is not limited to, Acute Flaccid Myelitis, Anaplasmosis, Anthrax, Babesiosis, Botulism, Brucellosis, Campylobacteriosis, Carbapenem-resistant Infection (CRE/CRPA), Chancroid, Chickenpox, Chikungunya Virus Infection (Chikungunya), Chlamydia, Ciguatera (Harmful Algae Blooms (HABs)), Clostridium Difficile Infection, Clostridium Perfringens (Epsilon Toxin), Coccidioidomycosis fungal infection (Valley fever), COVID-19 (Coronavirus Disease 2019), Creutzfeldt-Jacob Disease, transmissible spongiform encephalopathy (CJD), Cryptosporidiosis (Crypto), Cyclosporiasis, Denguc, 1,2,3,4 (Dengue Fever), Diphtheria, E. coli infection, Shiga toxin-producing (STEC), Eastern Equine Encephalitis (EEE), Ebola Hemorrhagic Fever (Ebola), Ehrlichiosis, Encephalitis, Arboviral or parainfectious, Enterovirus Infection, D68 (EV-D68), Enterovirus Infection, Non-Polio (Non-Polio Enterovirus), Giardiasis (Giardia), Glanders, Gonococcal Infection (Gonorrhea), Granuloma inguinale, Haemophilus Influenza disease, Type B (Hib or H-flu), Hantavirus Pulmonary Syndrome (HPS), Hemolytic Uremic Syndrome (HUS), Hepatitis (A, B, C, D, and/or E), Herpes Herpes Zoster, zoster VZV (Shingles), Histoplasmosis infection (Histoplasmosis), Human Immunodeficiency Virus/AIDS (HIV/AIDS), Human Papillomavirus (HPV), Influenza (Flu), Lead Poisoning, Legionellosis (Legionnaires Disease), Leishmaniasis, Leprosy (Hansens Disease), Leptospirosis, Listeriosis (Listeria), Lyme Disease, Lymphogranuloma venereum infection (LGV), Malaria, Measles, Melioidosis, Meningitis, Viral (Meningitis, viral), Meningococcal Disease, Bacterial (Meningitis, bacterial), Middle East Respiratory Syndrome Coronavirus (MERS-COV), Mononucleosis, Multisystem Inflammatory Syndrome in Children (MIS-C), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan, Psittacosis (Parrot Fever), Phthiriasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella, Salmonellosis gastroenteritis (Salmonella), Scabies Infestation (Scabies), Scombroid, Septic Shock (Sepsis), Severe Acute Respiratory Syndrome (SARS), Shigellosis gastroenteritis (Shigella), Smallpox, Staphylococcal Infection, Methicillin-resistant (MRSA), Staphylococcal Food Poisoning, Enterotoxin-B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A (invasive)), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus, Toxoplasmosis, Trichomoniasis (Trichomonas infection), Trichinosis Infection (Trichinosis), Tuberculosis (Latent) (LTBI), Tuberculosis (TB), Tularemia (Rabbit fever), Typhus, Typhoid Fever, Group D, Vaginosis, bacterial (Yeast Infection), Vaping-Associated Lung Injury (e-Cigarette Associated Lung Injury), Varicella (Chickenpox), Vibrio cholcrac (Cholera), Vibriosis (Vibrio), Viral Hemorrhagic Fever (Ebola, Lassa, Marburg), West Nile Virus, Yellow Fever, Yersenia (Yersinia), or Zika Virus Infection (Zika).

In some embodiments, when the sample is obtained from a patient, the patient has been diagnosed with, or is suspected to be suffering from an infection caused by a bacterium selected from the group consisting of: Acinetobacter, Actinomyces, Aerococcus, Bacteroides, Bartonella, Brucella, Bordetella, Burkholderia, Campylobacter, Chlamydia, Citrobacter, Clostridium, Corynebacterium, Edwardsiella, Elizabethkingia, Enterobacter, Enterococcus, Escherichia, Fusobacterium, Haemophilus, Helicobacter, Klebsiella, Legionella, Leptospira, Listeria, Morganella, Mycobacterium, Mycoplasma, Neisseria, Pantoea, Prevotella, Proteus, Providencia, Pseudomonas, Raoultella, Salmonella, Serratia, Shigella, Staphylococcus, Stenotrophomonas, Streptococcus, Ureaplasma, and Vibrio.

In some embodiments, when the sample is obtained from a patient, the patient has been diagnosed with, or is suspected to be suffering from an infection caused by a virus selected from the group consisting of: bacteriophage, RNA bacteriophage (e.g., MS2, AP205, PP7 and QB), Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, and Poliovirus, Norovirus, Zika virus, Dengue Virus, Rabies Virus, Newcastle

### Disease Virus, and White Spot Syndrome Virus.

In some embodiments, when the sample is obtained from a patient, the patient has been diagnosed with, or is suspected to be suffering from an infection caused by a parasite selected from the group consisting of: Plasmodium, Trypanosoma, Toxoplasma, Giardia, Leishmania, Cryptosporidium, helminthic parasites: Trichuris spp., Enterobius spp., Ascaris spp., Ancylostoma spp, and Necatro spp., Strongyloides spp., Dracunculus spp., Onchocerca spp, and Wuchereria spp., Taenia spp., Echinococcus spp., and Diphyllobothrium spp., Fasciola spp., and Schistosoma spp.

### Pathogen

In some embodiments, a pathogen is a microorganism or microbial fragment. In some embodiments, the pathogen may comprise a whole (infectious) pathogen cell, or a part of the pathogen cell, such as, a cell wall component of a microorganism. In some embodiments, the pathogen comprises a pathogen fragment, a pathogen debris, a pathogen nucleic acid, a pathogen lipoprotein, a pathogen surface glycoprotein, a pathogen membrane component, or a component released from the pathogen. In some embodiments, the pathogen is, is derived from, or is isolated from bacteria, fungus, prokaryote, virus, phage, or a misfolded protein (e.g., a prion).

In some embodiments, the pathogen is a disease-causing microorganism selected from the group consisting of a bacterium, a fungus, a virus, an archaea, and a parasite, or a fragment thereof.

In some embodiments, the pathogen is a disease-causing microorganism that causes a urogenital, skin, lung, or gastrointestinal disease. In some embodiments, the pathogen is a disease-causing microorganism that causes a urogenital disease, including but not limited to, bacterial vaginosis, vulvovaginal candidiasis, sexually transmitted diseases, or urinary tract infections. In some embodiments, the pathogen is a disease-causing microorganism that causes a skin disease, including but not limited to, atopic dermatitis, acne, methicillin-resistant Straphylococcus aureus (MRSA), or epidermolysis bullosa. In some embodiments, the pathogen is a disease-causing microorganism that causes a lung disease, including but not limited to, cystic fibrosis, Mycobacterium tuberculosis, or pneumonia. In some embodiments, the pathogen is a disease-causing microorganism that causes a gastrointestinal disease, including but not limited to stomach ulcer, ulcerative colitis, Crohn's disease, irritable bowel syndrome, Clostridioides difficile infection, celiac's disease, lactose intolerance, food allergies, small intestine bacterial overgrowth, viral gastroenteritis, necrotizing enterocolitis, or gingivitis.

### Microbial Strain

In some embodiments, the at least one microbial strain is selected from the group consisting of a bacterium, a synthetic bacterium, a synthetic organism, a fungus, a virus, an archaea, a parasite, and a genetically modified organism (e.g., wherein specific genes are added, deleted, mutated, driven to high expressions, and/or suppressed to low expression levels). In some embodiments, the at least one microbial strain is a bacterial strain. In some embodiments, the bacterial strain is a probiotic strain. In some embodiments, the at least one microbial strain is a genetically engineered strain. In some embodiments, the at least one microbial strain is a genetically engineered bacterial strain. In some embodiments, the genetically engineered strain is a genetically engineered bacterial strain. In a “genetically engineered” strain, specific genes are added, deleted, mutated, driven to high expressions, and/or suppressed to low expression levels. The alterations are known to a person of ordinary skill in the art.

### Encoding Probe Hybridization

In the methods described herein, the method can include contacting the at least one encoding probe with the first complex to form a second complex, wherein the at least one encoding probe comprises a targeting sequence and a landing pad sequence. This step may also be referred to as the “encoding probe hybridization” step. In here, at least one encoding probe is contacted with the sample to produce a second complex. The second complex can include the targeting sequence of the encoding probe hybridized to a nucleic acid target sequence. In some embodiments, the nucleic acid target sequence is a sequence present in the pathogen or the at least one microbial strain.

In some embodiments, contacting the encoding probes with the sample is contacting the encoding probes with at least one nucleotide sequence of the sample. In some embodiments, contacting the encoding probes with the sample is hybridizing the encoding probe (e.g., via the targeting sequence present in the encoding probe) with a target sequence present in the sample.

In some embodiments, in order to contact the encoding probes with the sample, the sample can be digested or lysed so as to allow the encoding probes (and other probes described herein) to contact with the target sequence.

Encoding probes are probes that bind directly to a target or targeting sequence and contain either one or two branches extending away from the hybridization site. The branches can either correspond to the readout sequences. Encoding probes, for example, are designed to target bacterial ribosomal RNA (rRNA) and messenger RNA (mRNA) targets.

For example, rRNA-probes can contain (5′ to 3′):
a. Primer sequences to enrich probe pool.
b. A readout-complementary sequence.
c. rRNA target complementary sequence.
d. A readout-complementary sequence (can be same or different than b).
e. Primer sequences to enrich probe pool.

mRNA-probes contain (5′ to 3′):
a. Primer sequences to enrich probe pool.
b. An initiator sequence.
c. mRNA target complementary sequence.
d. An initiator sequence (can be same or different than b).
e. Primer sequences to enrich probe pool.

### Primer Sequences

In some embodiments, the primer sequence can include about 10 to about 30, about 15 to about 25, about 18 to about 23, about 15, 16, 17, 18, 19, 20, 21, 22, or 23 nucleotides long.

### Targeting Sequence

In some embodiments, the targeting sequence targets at least one of messenger RNA (mRNA), micro RNA (miRNA), long non coding RNA (lncRNA), ribosomal RNA (rRNA), small interfering RNA (siRNA), transfer RNA (RNA), Crispr RNA (crRNA), trans-activating cirspr RNA (tracrRNA), mitochondria RNA, Intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigenic target. In some embodiments, the target is mRNA. In some embodiments, the target is rRNA. In some embodiments, the target is mRNA and rRNA.

In some embodiments, the targeting sequence targets mRNA and/or rRNA. In some embodiments, the targeting sequence targets mRNA. In some embodiments, the targeting sequence targets rRNA. In some embodiments, the targeting sequence targets a 16S rRNA sequence, a 5S IRNA sequence, and/or a 23S rRNA sequence in the microbial strain. In some embodiments, the targeting sequence targets a 16S rRNA sequence, a 5S rRNA sequence, and/or a 23S rRNA sequence in a bacterial strain. In some embodiments, the targeting sequence targets a 16S rRNA sequence, a 5S rRNA sequence, and/or a 23S rRNA sequence in the pathogen.

In some embodiments, the targeting sequence of the encoding probe is substantially complementary to a specific target sequence. By “substantially complementary” it is meant that the nucleic acid fragment is capable of hybridizing to at least one nucleic acid strand or duplex even if less than all nucleobases do not base pair with a counterpart nucleobase.

In some embodiments, to hybridize the encoding probes to the first complex, encoding buffer is added to the sample. In some embodiments, the encoding buffer can include a denaturing/deionizing agent, a salt buffer, a detergent, a polyanionic polymer, a blocking agent, or combinations thereof. In some embodiments, the encoding buffer includes more than one type of agent, for example, the encoding buffer can include two or more polyanionic polymers and/or two or more blocking agents.

### Readout Probe Hybridization

After the encoding hybridization step is complete, emissive readout probes are added to the second complex, wherein each emissive readout probe can include a label and a complementary sequence to the readout sequence of the encoding probe. In some embodiments, this step may be referred to as the “readout probe hybridization” step. In here, the emissive readout probes hybridize to their complementary sequences present in the second complex.

Emissive readouts probes are 10-50 nucleotide-long oligonucleotides bound with one of ten fluorescent dyes at the 5′- and/or 3′-end.

Readout probes can be designed as follows:
a. Are coupled to 1, 2, or more fluorescent dyes.
b. Are orthogonal to all biological sequences.
c. Are orthogonal to each other/each other's complementary sequences.

In some embodiments, the emissive readout sequence is about 10 to about 50, about 15 to about 50, about 15 to about 45, about 15 to about 35, about 15 to about 30, about 18 to about 24, about 10, 11, 12, 13, 14, 15, 16, 17, 18, 19, 20, 21, 22, 23, or 24 nucleotides long.

In some embodiments, the emissive readout probe can include a label on the 5′ or 3′ end. In some embodiments, the emissive readout probe can include a label on the 5′ end and a label on the 3′ end. In some embodiments, the labels are the same. In some embodiments, the labels are different.

In some embodiments, the label is a fluorescent entity (fluorophore) or phosphorescent entity. In some embodiments, the label is a cyanine dye (e.g., Cy2, Cy3, Cy3B, Cy5, Cy5.5, Cy7, etc.), Alexa Fluor dye, Atto dye, photoswtichable dye, photoactivatable dye, fluorescent dye, metal nanoparticle, semiconductor nanoparticle or “quantum dots”, fluorescent protein such as GFP (Green Fluorescent Protein), or photoactivatable fluorescent protein, such as PAGFP, PSCFP, PSCFP2, Dendra, Dendra2, EosFP, tdEos, mEos2, mEos3, PAmCherry, PAtagRFP, mMaple, mMaple2, and mMaple3.

In some embodiments, the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

In some embodiments, to hybridize the readout probes to the second complex, readout buffer is added to the sample. In some embodiments, the readout buffer can include a denaturing/deionizing agent, a salt buffer, a detergent, a polyanionic polymer, a blocking agent, or combinations thereof. In some embodiments, the readout buffer includes more than one type of agent, for example, the readout buffer can include two or more polyanionic polymers and/or two or more blocking agents.

In some embodiments, after each reaction and before proceeding to the next one, the samples or probes are washed with a “wash buffer.”

In some embodiments, the wash buffer can include a denaturing/deionizing agent, a salt buffer, a detergent, a polyanionic polymer, acids, a pH stabilizer, a chelating agent, or combinations thereof. In some embodiments, the wash buffer can include more than one type of agent, for example, the wash buffer can include two or more detergents. In some embodiments, the wash buffer can include a denaturing/deionizing agent, a salt buffer, a detergent, a polyanionic polymer, and an acid. In some embodiments, the wash buffer can include a salt buffer and a detergent. In some embodiments, the wash buffer can include a salt buffer, a pH stabilizer, and a chelating agent.

In some embodiments, the label is imaged using widefield epifluorescence microscopy, widefield phase contrast microscopy, point scanning confocal microscopy, spinning disk confocal microscopy, lattice lightsheet microscopy, or light field microscopy.

In some embodiments, specimens imaged with the above modalities can be imaged at different spatial resolution using different microscope objectives including 10×, 20×, 32×, 40×, 63×, or 100×, which can be compatible with air, oil, or water immersion medium.

In some embodiments, the detection strategy used is channel, spectral, channel and fluorescence lifetime, or spectral and fluorescence lifetime.

In some embodiments, the labels used in the present methods are imaged using a microscope. In some embodiments, the microscope is a confocal microscope. In some embodiments, the microscope is a fluorescence microscope. In some embodiments, the microscope is a light-sheet microscope. In some embodiments, the microscope is a super-resolution microscope.

In some embodiments, the transcripts within the labels are identified using spot segmentation and classification.

In some embodiments, the host cells are identified by analyzing their transcripts profile.

In some embodiments, the host cells and microbial strains are imaged and identified simultaneously.

In some embodiments, the sample is on an analyzing platform, wherein the analyzing platform is a microscope slide, at least one chamber, at least one microfluidic device, at least one well, at least one plate, or at least one filter membrane.

### Constructs and Libraries

In another aspect of the present disclosure provides a library of constructs, each construct comprising:
(a) a target cell;
(b) a first plurality of encoding and readout probes, wherein each encoding probe comprises a targeting sequence that is a region of interest on a nucleotide of a pathogen and a landing pad sequence; and each readout probe comprises a label and a sequence complementary to the landing pad sequence; and
(c) a second plurality of encoding and readout probes, wherein each encoding probe comprises a targeting sequence that is a region of interest on a nucleotide of a microbial strain and a landing pad sequence; and each readout probe comprises a label and a sequence complementary to the landing pad sequence.

In another aspect, the present disclosure provides a library of constructs, where each construct can comprise:
(a) a target cell;
(b) a first plurality of encoding and readout probes, wherein each encoding probe comprises a targeting sequence that is a region of interest on a nucleotide of a pathogen and a landing pad sequence; and each readout probe comprises a label and a sequence complementary to the landing pad sequence; and
(c) a second plurality of encoding and readout probes, wherein each encoding probe comprises a targeting sequence that is a region of interest on a nucleotide of a bacterial strain and a landing pad sequence; and each readout probe comprises a label and a sequence complementary to the landing pad sequence.

### Spatial Location

In another aspect, the present disclosure provides methods for determining the spatial location of a complex, comprising:
(i) providing:
   (a) a target cell;
   (b) a pathogen;
   (c) at least one microbial strain, and
   (d) at least one set of probes, wherein the at least one set of probes comprises at least one encoding probe and at least one emissive readout probe;
(ii) performing an imaging assay, wherein the imagining assay comprises:
   (A) contacting the (a) target cell with the (b) pathogen and the (c) at least one microbial strain to produce a first complex;
   (B) contacting the at least one encoding probe with the first complex to form a second complex, wherein the at least one encoding probe comprises a targeting sequence and a landing pad sequence,
   (C) contacting the at least one emissive readout probe with the second complex, wherein the at least one emissive readout probe comprises a label and a sequence complementary to the landing pad sequence;
   (D) acquiring one or more emission spectra from the at least one emissive readout probe;
   (E) repeating the aforementioned steps for at least one different encoding probe;
   (F.1) determining the spectra of “signal” and assigning them to a species of the at least one microbial strain;
   (F.2) determining the spectra of “signal” and assigning them to a species of the pathogen;
   (G) decoding the spectra into a single, targeted transcript through means of signal deconvolution, error correction, comparison to reference standards;
   (H) performing a second assay to image the target cell; and
(iii) quantify the abundance of the at least one microbial strain to the target cell and/or the association between the pathogen to the target cell, thereby determining the spatial location of the complex, wherein the complex is a target cell-microbial strain complex or a target cell-pathogen complex.

In another aspect, the present disclosure provides a method for determining the spatial location of a complex, the method can comprise:
(i) providing:
   (a) a target cell;
   (b) a pathogen;
   (c) at least one bacterial strain, and
   (d) at least one set of probes, wherein the at least one set of probes comprises at least one encoding probe and at least one emissive readout probe;
(ii) performing an imaging assay, wherein the imagining assay comprises:
   (A) contacting the (a) target cell with the (b) pathogen and the (c) at least one bacterial strain to produce a first complex;
   (B) contacting the at least one encoding probe with the first complex to form a second complex, wherein the at least one encoding probe comprises a targeting sequence and a landing pad sequence,
   (C) contacting the at least one emissive readout probe with the second complex, wherein the at least one emissive readout probe comprises a label and a sequence complementary to the landing pad sequence;
   (D) acquiring one or more emission spectra from the at least one emissive readout probe;
   (E) repeating the aforementioned steps for at least one different encoding probe;
   (F.1) determining the spectra of “signal” and assigning them to a species of the at least one bacterial strain;
   (F.2) determining the spectra of “signal” and assigning them to a species of the pathogen;
   (G) decoding the spectra into a single, targeted transcript through means of signal deconvolution, error correction, comparison to reference standards;
   (H) performing a second assay to image the target cell; and
(iii) quantify the abundance of the at least one bacterial strain to the target cell and/or the association between the pathogen to the target cell, thereby determining the spatial location of the complex,
wherein the complex is a target cell-bacterial strain complex or a target cell-pathogen complex.

### Barcoded Probes

The encoding probes used in the methods described herein, constructs and libraries described herein use barcoded probes. The barcoded probes represent a probe/sequence that is specific to a target sequence in the sample/complex with a unique code.

In some embodiments, the barcoded probes include the encoding probes and readout sequences described herein.

In some embodiments, each sample or target in the sample to be identified is assigned a unique n-bit binary code selected from a plurality of unique n-bit binary codes, where n is an integer greater than 1.

A “binary code” refers to a representation of target sequence in a sample using a string made up of a plurality of “0” and “1” from the binary number system. The binary code is made up of a pattern of n binary digits (n-bits), where n is an integer representing the number of labels used. The bigger the number n, the greater number of targets can be represented using the binary code. For example, a binary code of eight bits (an 8-bit binary code, using 8 different labels) can represent up to 255 (2⁸-1) possible targets. (One is subtracted from the total possible number of codes because no target sequence is assigned a code of all zeros “00000000.” A code of all zeros would mean no decoding sequence, and thus no label, is attached. In other words, there are no non-labeled target sequences.) Similarly, a binary code of ten bits (a 10-bit binary code) can represent up to 1023 (2¹⁰-1) possible target sequences. In some embodiments a binary code may be translated into and represented by a decimal number. For example, the 10-bit binary code “0001100001” can also be represented as the decimal number “97.”

Each digit in a unique binary code represents whether a readout probe and the fluorophore corresponding to that readout probe are present for the selected species. In some embodiments, each digit in the binary code corresponds to a Readout probe (from Readout probe 1 (R1) through Readout probe n (Rn) in an n-bit coding scheme). In a specific embodiment, the n is 10 and the digits of an n-bit code correspond to R1 through R10. In some embodiments, the fluorophores that correspond to R1 through Rn are determined arbitrarily. For example, n is 10, and R1 corresponds to an Alexa 488 fluorophore, R2 corresponds to an Alexa 546 fluorophore, R3 corresponds to a 6-ROX (6-Carboxy-X-Rhodamine, or Rhodamine Red X) fluorophore, R4 corresponds to a PacificGreen fluorophore, R5 corresponds to a PacificBlue fluorophore, R6 corresponds to an Alexa 610 fluorophore, R7 corresponds to an Alexa 647 fluorophore, R8 corresponds to a DyLight-510-LS fluorophore, R9 corresponds to an Alexa 405 fluorophore, and R10 corresponds to an Alex532 fluorophore. In some embodiments, other labels/fluorophores are used in the n-bit encoding system.

In some embodiments, the n-bit binary code is selected from the group consisting of 2-bit binary code, 3-bit binary code, 4-bit binary code, 5-bit binary code, 6-bit binary code, 7-bit binary code, 8-bit binary code, 9-bit binary code, 10-bit binary code, 11-bit binary code, 12-bit binary code, 13-bit binary code, 14-bit binary code, 15-bit binary code, 16-bit binary code, 17-bit binary code, 18-bit binary code, 19-bit binary code, 20-bit binary code, 21-bit binary code, 22-bit binary code, 23-bit binary code, 24-bit binary code, 25-bit binary code, 26-bit binary code, 27-bit binary code, 28 bit binary code, 29-bit binary code, and 30-bit binary code.

The methods and constructs described herein have significant advantages of those currently available in the art.

### EXAMPLES

The screening methods described herein can be used by utilizing atopic dermatitis model. By introducing S. aureus to human keratinocyte monolayers, we can simulate S. aureus-associated atopic dermatitis.

### Example 1. Characterize a Complex LBP Using a Low Magnification Platform with Fast Turnaround Times

Experimental Design: Microbiomes are defined by complex interactions from many species. In an effort to screen LBPs with similar complexity, we will construct LBP consortia using 25 strains from the healthy skin microbiome, such as Cutibacterium acnes and Micrococcus luteus, for therapeutic value against disease-associated S. aureus (NCTC 13434). We will verify imaging accuracy and compare low- and high-magnification imaging modalities while measuring adherent microbiomes for each keratinocyte. Low-magnification imaging success will be indicated by high concordance with high-magnification imaging (Spearman correlation R²>0.90) and a four-fold data acquisition rate increase.

Experimental Method: We will acquire strains from ATCC, NCTC/Microbiologics, or from stocks present in our laboratory. For each strain, we will validate 16S rRNA sequence by Oxford Nanopore MinIon sequencing of extracted rRNA. We will design a probe panel for each species's 16S rRNA with a unique combination of two fluorophores (from 8 total). S. aureus will be encoded with a single, separate fluorophore (Alexa 647). Strains will be cultured, fixed, and assayed for specificity. A unique Alexa 405-encoded Eubacterium probe will verify the total bacteria count, enabling us to assess detection sensitivity. We will determine the maximum tolerable dose (MTD) for each strain in HEKa keratinocytes (Invitrogen, Carlsbad, CA). Beginning with 10⁹ CFU/mL, ten-fold dilutions of each strain will be added to keratinocytes for 2 hours. Cellular viability will be assessed using a Trypan Blue exclusion test, and the highest dose maintaining >95% viability compared to control will be defined as the MTD for each strain.

To test probe specificity in vitro, LBP strains at their MTDs will be pooled, introduced to keratinocyte monolayers, and incubated for one hour. After washing and fixing, we will perform our standard assay, collect low-(3 FOVs per well) and high-magnification (9 FOVs per well) images, and identify all microbial cells with 3 μm of keratinocytes for three unique wells. The microbial abundance vectors from low- and high-magnification images will be compared using Spearman correlation.

Expected outcomes: We will produce a single encoding probe panel capable of measuring 28 LBP candidate species and S. aureus, representing an order of magnitude more complexity than past LBP screening systems. We will validate the speed and accuracy of the low-magnification platform, thereby enabling rapid screening. We will denoise the images to improve classification accuracy. If imaging at 20× yields poor accuracy, we can proceed to future aims using other objectives (e.g., 32× or 40×) that still offer faster imaging.

### Example 2. Evaluate Engraftment of S. aureus with LBP Treatment

Experimental Design: The value of screening drug candidates is in sampling many combinations, iterations, or modalities to quickly identify components that maximize therapeutic effect. We will use our high-throughput screening platform to examine 50 combinations of up to 5 species (randomly chosen from 25) for protecting keratinocytes against adhesion from two different S. aureus strains. We will compare each host cell's specific adherent microbiome to other candidates and controls. This data will identify potential therapeutic species, and we will rank consortia based on their effectiveness in reducing adherent S. aureus cells compared to controls.

Experimental Methods: We will have two strains of S. aureus (one with pathogenic association to serious skin disease (NCTC 13434) and one from a healthy individual (NCTC 14139)) and, for each strain, determine their MTD in the presence of keratinocytes for 2 hours. For each LBP consortium, we will dilute species to their MTD and mix up to five strains at equal volume. We will perform experiments in a 12-well plate format, growing keratinocyte monolayers until they reach 90% confluence and then adding the LBP consortia with S. aureus at 25% of its MTD (determined as in Aim 1). For each 12-well plate, we will assay an S. aureus strain against nine different LBP consortia; two wells will be reserved for S. aureus-only positive controls and one well will serve as a negative control, without microbiota. Single-species conditions (i.e. LBP species competing against S. aureus) will also be evaluated.

For each well, we will acquire one low-magnification FOV from the center of the specimen well. We will process the images using our standard pipeline, and attribute a local, adherent microbiome to each host cell which includes the number of S. aureus bacteria. The number of adherent S. aureus will be calculated as those with >50% of their cellular area within 3 μm of the cytoplasm boundary. For each LBP, we will calculate a protection score, PS, such that:

$$P S = 1 - \frac{2 \underline{l}}{\underline{{p}_{1}} + \underline{{p}_{2}}} ,$$

where l is the total number of adherent S. aureus bacteria divided by the total number of keratinocytes in a FOV assigned to an LBP (average number of S. aureus bacteria per keratinocyte), and p_{1,2} are the average number of S. aureus bacteria per keratinocyte in the positive controls. A PS of 1 would indicate a complete protection from S. aureus adhesion. We will assay each LBP candidate in triplicate; those with a coefficient of variation (CV) over 20% will be flagged and dropped from further analysis.

Expected outcomes: We will determine the potential therapeutic value of each LBP consortia and determine species that typically yield reduced S. aureus adhesion. Complex LBPs will be compared to single species conditions to examine synergistic effects between species across conditions. These findings will show an order of magnitude in the throughput of LBP candidate selection compared to other studies. To prevent contamination from skin microorganisms, scientists will wear protective equipment, including lab coats, elbow-length nitrile gloves, and face shields. Cell culture will be conducted in a decontaminated Class II Biosafety Cabinet after each use.

### Example 3. Evaluate the Innate Immune Response after LBP Treatment for 25 Complex Candidates

Experimental Design: To evaluate the therapeutic potential of LBPs in vivo, it is crucial to understand their effect on keratinocyte populations. Microbes may cause an inflammatory response that exacerbates atopic dermatitis and promotes further disease. Our ability to detect microbiota and RNA transcripts in tissue specimens will provide valuable insights to customers seeking to understand the therapeutic effects of their LBP candidates. We will measure the expression of seven host genes across keratinocytes which have been validated previously in literature and examine the changes in gene expression for the consortia with the greatest effect on S. aureus (Example 2). We will report the differential expression of each consortium over unperturbed cells and highlight the three consortia that show the greatest reduction in S. aureus abundance with the lowest differential expression of host genes.

Experimental Methods: We will design a host gene expression panel to detect keratinocyte-specific (KRT16, ANXA8) and immune-response (CXCL2, IL6, IL8, IL13, NF-κB) genes. We will examine the signal generated by each individual gene when keratinocytes are unperturbed and when they are exposed to a pathogen at various concentrations to validate immune-response gene detection. We will use a 12-well plate setup to profile 25 LBP consortia (without S. aureus) on a plate similar to Example 2. We will examine the 25 consortia with the highest protection scores from Example 2.

After incubating LBP consortia bound to keratinocytes, performing our mRNA-detection assay (as described in PCT Application No.: PCT/US2023/062917 and U.S. application Ser. No. 18/171,850; each incorporated herein by reference in their entireties), and imaging (one field of view at low magnification), we will compile an adherent microbiome profile (similar to Example 1) and a gene expression profile for each host cell. For each consortium, we will report the mean expression of each gene (via summed intensity of each gene in each host cell) and compare it to control samples. LBPs with potential therapeutic value will generate broad detection from S. aureus in Example 2 while also leading to low expression of immune-response-related genes.

Expected outcomes: We will show the host effect for LBP consortia with the greatest pathogen reduction. Including host cell measurements will demonstrate our platform can be used to show safety and therapeutic effect of LBPs.

If imaging with a 20× objective does not accurately determine microbial abundances, a higher magnification data acquisition platform can be used.

Feasibility can be successfully determined if the probe panel can identify bacterial species with >95% accuracy and >99% sensitivity, and if the platform can report LBP candidates that reduce S. aureus adhesion by >50% and limit innate immune response gene expression to 50% baseline values.

### Example 4. Evaluate the Reduction in Gardnerella vaginalis Adhesion to Vaginal Epithelial Cells with Interference by LBP Candidates

Experimental Design: Bacterial vaginosis is primarily caused by colonization and biofilm formation by G. vaginalis after microbiome dysbiosis. We will model the use of an LBP therapeutic in an interference assay. LBP candidates representing species from the healthy vaginal microbiome (e.g. Lactobacillus spp., Actinobacteria, and Prevotella) will be sourced from biobanks and cultured in our laboratory. Complex mixtures of 3 to 12 species will be generated and added below their minimum tolerable dose to vaginal epithelial cells. After LBP candidate adhesion, G. vaginalis cells will be added at their minimum tolerable dose. We will compare the average epithelial cell microbiome in cases of LBP introduction before pathogen to pathogen only controls to measure the reduction in adhesion.

Experimental Methods: Vaginal epithelial cells (VEC; acquired from ATCC, PCS-480-010) will be cultured at recommended conditions until they reach 90% confluency in wells of a glass-bottom, 12-well tissue culture plate. LBP candidates will be generated randomly, by selecting 3 to 12 species, diluting to their minimum tolerable dose, and mixing in equal volumes. The LBP species will be incubated with the VECs for one hour. VECs will be washed five times with fresh media to remove nonadherent or weakly adherent cells. G. vaginalis will be added at its minimum tolerable dose and incubated with VECs for one hour. VECs will be washed five times with fresh media to remove nonadherent or weakly adherent cells. All cells will be fixed in 2% formaldehyde and we will apply an species-specific LBP panel using the HiPR-FISH assay and staining cells with DAPI.

After staining, we will image specimens on the confocal microscope (Zeiss i880 or 1990) and both VECs and microbial cells are segmented and, for microbial cells, classified. For each VEC, we then record the number of each species in the panel, including G. vaginalis, within a 3 micron proximity. For 50 randomly generated LBPs, we will compare the protection score to identify candidates that support strong repulsion of G. vaginalis.

Expected outcomes: We will determine the species (both individually and in 50 LBPs) that generate the strongest protect to G. vaginalis in VECs.

### Example 5. Biological Change Caused by Introduction of Gardnerella vaginalis to Vaginal Epithelial Cells

Methods: 50 μg/mL poly-D-lysine was added to coated chambered slides for 1 hour at room temperature to functionalize the surface for vaginal epithelial cell adherence. The chambered slides were washed three times with 1×PBS and air dried in a sterile hood before use. Primary vaginal epithelial cells (VECs), suspended in ReproLife™ Reproductive medium, were added into a chamber (30,000 VECs) per well under sterile conditions. The chambered cover slides were placed in secondary containment (Petri dishes) and incubated at 5% CO₂ conditions at 37° C. for three days. Separately, Gardnerella vaginalis, Lacticaseibacillus rhamnosus, and Lactobacillus acidophilus were grown to log phase. Bacterial suspensions were centrifuged at 4000 rpm for five to ten minutes at room temperature. After removing the supernatant, VEC media without antibiotics was added to wash bacteria. A second round of centrifugation (5 to 10 minutes at 4000 rpm) was performed and the microbes were resuspended to their desired concentration.

Each well was inoculated with either Gardnerella vaginalis, Lacticaseibacillus rhamnosus, or Lactobacillus acidophilus, by first removing the media on the VEC cells and then adding the bacteria resuspended in VEC media. 500 μL of VEC media consisting of 10⁸ bacterial cells was added and incubated for 1 hour at 37° C. At the conclusion of 1 hour, the VEC cells were washed three times (by removing the current VEC media and then adding fresh, antibiotic-free media) to remove non-adherent bacteria.

To collect cells for RNA-seq measurements, VEC media was removed from each well and 250 μL of 1×PBS was added. A sterile, bent pipette tip was used to scrape off VEC cells from the monolayer. The 1×PBS with scraped VECs was transferred to a 1.5 mL Eppendorf tube. The cell suspension was centrifuged at 500×g for 5 minutes and the supernatant was removed.

RNA extraction was prepared by using a Zymo RNA Miniprep Kit. Cells were washed once with ice cold 1×PBS and then TriReagent was added to each tube and thoroughly mixed. Ethanol was added to each tube, transferred to a collection column and centrifuged at 15000×g for 30 seconds. The RNA was eluted from the column, transferred to a new tube, and treated with DNAse I to remove genomic DNA. The RNA was recovered, washed, and concentrated in DNAse/RNAse-free water. RNA concentration was calculated using the Qubit system.

DNA sequencing libraries were prepared using the Illumina Stranded Total RNA Prep, Ligation with Ribo-Zero Plus kit with 15 cycles of PCR amplification. DNA concentration was calculated using the Qubit system. Sequencing libraries were assessed for quality using 1% E-gel EX. Libraries were run on Illumina Miniseq with a High output 150-cycle kit with the following cycle conditions: R1—76 cycles, R2—76 cycles, I1—10 cycles, I2—10 cycles.

Data was processed through a proprietary sequencing analysis pipeline that involved: (1) checking the quality of the sequencing of the reads, and removing low quality reads, (2) aligning transcripts to the human transcriptome, and (3) summarizing the transcript counts and determining gene ontology (GO).

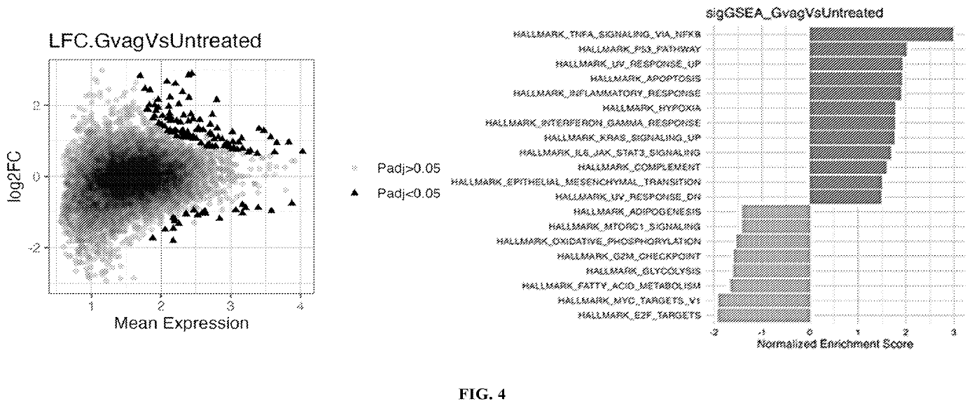

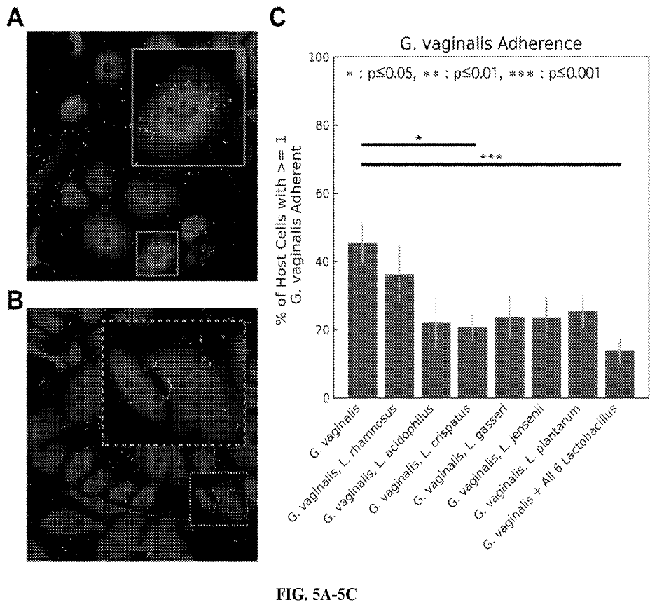

Results: Gene expression comparison revealed a significant increase in genes related to inflammatory processes. Enriched genes included those related to Nfkb signaling, inflammation, and apoptosis, while healthy cell processes (fatty acid metabolism and cell cycle checkpoints) were suppressed). These results (FIG. 4) indicate that VEC host cells physiologically change in our assay in a quantifiable way.

### Example 6

Objective: To demonstrate the ability to distinguish between “treatment” (i.e. adding beneficial bacteria to block the adherence of a pathogen) and a pathogen-only control in terms of the number of adherent pathogenic bacteria.

Methods: A nearly confluent monolayer of human vaginal epithelial cells (VECs) was created by adding Geltrex basement membrane to 16-well chambered coverslips for 1 hour at 37° C. followed by an hour at room temperature. VECs were plated onto the 16 well chamber, attempting to plate 10,000 cells per well. The monolayers reached confluence over the next three to five days, and the VEC media was changed to fresh media with antibiotics every 2 days.

On the day of inoculation, bacteria were cultured to an OD of 0.6 (measured via ThermoFisher Nanodrop) and the bacteria were counted to determine the concentration. The bacteria were centrifuged at 4500 rpm for 5 mins and resuspended in VEC media without antibiotics to wash away dead bacterial cells and bacterial media. The bacterial suspensions were again centrifuged for 5 minutes and resuspended in VEC media without antibiotics to a final concentration of 10⁸ bacterial cells per milliliter.

Table 1 shows each species used in the experiments (acquired from ATCC):

| Species | Strain |
|---|---|
| Lactobacillus crispatus | VPI 3199 |
| Lactobacillus jensenii | 62G |
| Lacticaseibacillus | BUCSAV 227 |
| rhamnosus | |
| Lactobacillus gasseri | DSM 20243 |
| Lactobacillus acidophilus | Scav |
| Lactobacillus plantarum | Lp 39 |
| Gardnerella vaginalis | 594 |

The adhesion experiments were set up as an interference assay, with the following conditions:
I. VECs with no microbiota added
II. VECs with 25 million Gardnerella vaginalis bacterial cells added
III. VECs with 25 million Lactobacillus acidophilus bacterial cells added
IV. VECs with 25 million Lacticaseibacillus rhamnosus bacterial cells added
V. VECs with 25 million Lactobacillus plantarum bacterial cells added
VI VECs with 25 million Lactobacillus crispatus bacterial cells added
VII. VECs with 25 million Lactobacillus gasseri bacterial cells added
VIII. VECs with 25 million Lactobacillus jensenii bacterial cells added
IX. VECs with 25 million Gardnerella vaginalis bacterial cells added, and then 25 million Lactobacillus acidophilus bacterial cells added
X. VECs with 25 million Gardnerella vaginalis bacterial cells added, and then 25 million Lacticaseibacillus rhamnosus bacterial cells added
XI. VECs with 25 million Gardnerella vaginalis bacterial cells added, and then 25 million Lactobacillus plantarum bacterial cells added
XII. VECs with 25 million Gardnerella vaginalis bacterial cells added, and then 25 million Lactobacillus crispatus bacterial cells added
XIII. VECs with 25 million Gardnerella vaginalis bacterial cells added, and then 25 million Lactobacillus gasseri bacterial cells added
XIV. VECs with 25 million Gardnerella vaginalis bacterial cells added, and then 25 million Lactobacillus jensenii bacterial cells added
XV. VECs with 25 million Gardnerella vaginalis bacterial cells added, and then a mixture of 4.2 million Lactobacillus acidophilus bacterial cells, 4.2 million Lacticaseibacillus rhamnosus bacterial cells, 4.2 million Lactobacillus plantarum bacterial cells, 4.2 million Lactobacillus crispatus bacterial cells, 4.2 million Lactobacillus gasseri bacterial cells, and 4.2 million Lactobacillus jensenii bacterial cells.

Each condition was repeated in replicate (4 or 6 replicates). Prior to inoculation the VECs with bacteria, VECs were washed with VEC media without antibiotics. VECs receiving bacteria (conditions II-XV) were inoculated with a total of 25 million bacterial cells. Slides were centrifuged in a swinging bucket rotor at 150×g for 5 minutes to promote adherence. The slides were then incubated for one hour at 37° C. in a 5% CO₂ incubator. Slides were then washed gently with antibiotic free, pre-warmed VEC media three times to remove non-adherent bacterial cells. Fresh VEC media without (conditions I to VIII) or with 25 million bacterial cells (conditions IX to XV) was added to VECs, slides were again centrifuged in a swinging bucket rotor at 150×g for 5 minutes to promote adherence and incubated for one hour at 37° C. in a 5% CO₂ incubator. After an hour, slides were then washed gently with antibiotic free, pre-warmed VEC media three times. After washing, VEC media was removed and replaced with freshly prepared 4% formaldehyde in 1×PBS so that it completely covered the monolayer. Cells were biologically fixed for 15 minutes at room temperature in this state. Then, fixed monolayers were washed twice with 1×DPBS and stored in 70% ethanol at 4° C.

To identify the bacteria at the species-level of taxonomic resolution, 70% ethanol was removed from the monolayers and they were washed twice in 2×SSC. We then added 200 μL of 10 mg/mL lysozyme to each sample for 30 minutes at 37° C. to permeabilize the cells. The samples were washed with 2×SSC, adding it for 15 minutes at room temperature, at the end of the incubation, the 2×SSC was completely aspirated. We added 50 μL of encoding buffer (2×SSC, 10% ethylene carbonate, 10% dextran sulfate, 5×Denhardt's solution, and 0.01% SDS) with probes and incubated the samples for 2 hours at 37° C. in a humidified chamber. After incubation, the encoding buffer was removed and replaced with 200 μL of wash buffer (215 mM NaCl, 20 mM Tris-HCl (pH 8.0), 5 mM EDTA) and incubated at 48° C. for 15 minutes to remove unbound probes. We then washed once with 2×SSC to remove residual wash buffer. We then added 50 μL of readout buffer (2×SSC, 10% ethylene carbonate, 10% dextran sulfate, 5×Denhardt's solution, and 0.01% SDS) with readout probes and incubated the wells in a dark, humidified chamber at room temperature for two hours. At the conclusion of the incubation, we repeated the wash step (wash buffer added for 15 minutes at 48° C. followed by a rinse with 5×SSC). We removed the buffers and added 20 ng/ml of DAPI (4′,6-Diamidino-2-Phenylindole, Dihydrochloride) in 5×SSC to each well for 3 minutes at room temperature. The DAPI was removed and replaced with 5×SSC for imaging.

Slide chambers were imaged on a confocal microscope (Zeiss i880, in lambda mode) with a 20× air objective. Samples were excited using 405 nm, 488 nm, 514 nm, 561 nm, and 633 nm laser and the emitted light was collected in 9 nm bins between the excitation wavelength and 700 nm. Data was processed to generate taxonomic identification for each microbe and assign them to cells identified by DAPI-staining of VEC nuclei.

Results: Following image processing, we created a profile of each VEC identified in our pipeline and all microbiota with 3-5 nM of the estimated cytoplasmic boundary. Each adherent microbe was attributed with a taxonomic identification of one out of our seven species or “unidentified”. We then examined the total number of adherent microbiota and the number of cells of each species adherent to each VEC. We determined that the addition of any Lactobacillus species after Gardnerella adhesion reduces the number of VECs with adherent Gardnerella. Additionally, a consortium of all Lactobacillus species outperforms any individual strain and reduced the fraction of Gardnerella-adherent VECs from one to two to less than 1 in 6. These results demonstrate the strength of our platform in determining the optimal conditions to achieve LBP effectiveness.

TABLE 2
Sequences used in Example 6.

| SEQ ID NO: | Description | Sequence |
|---|---|---|
| 1 | Encoding probe 1 | TGTGGAGGGATTGAAGGATATCCACCCACGCGTTACTCACCGT |
| | | ATGTGGAGGGATTGAAGGATA |
| 2 | Encoding probe 2 | TGTGGAGGGATTGAAGGATACGACGCCGCCTTTTAAACTTCTTT |
| | | CTACTGTGGAGGGATTGAAGGATA |
| 3 | Encoding probe 3 | TGTGGAGGGATTGAAGGATAACTGTTTCTGATGCAGTTCTTCGG |
| | | TACTTGTGGAGGGATTGAAGGATA |
| 4 | Encoding probe 4 | TGTGGAGGGATTGAAGGATACGGAGCGTCAGTTACAGACCAGA |
| | | GAGGGCTGTGGAGGGATTGAAGGATA |
| 5 | Encoding probe 5 | TGTGGAGGGATTGAAGGATAGTGCCTGTCTCTTTGTCCCCGAAC |
| | | CCTGTGGAGGGATTGAAGGATA |
| 6 | Encoding probe 6 | TGTGGAGGGATTGAAGGATATCGTAAGAGATCCGCTTGCCTTC |
| | | AGTCTGTGGAGGGATTGAAGGATA |
| 7 | Encoding probe 7 | TGTGGAGGGATTGAAGGATACCGCTGGCTCCAAAGGTTACCTC |
| | | AGGCTGTGGAGGGATTGAAGGATA |
| 8 | Encoding probe 8 | TGTGGAGGGATTGAAGGATAAAATGCGAAAGTAGCTTTTATCC |
| | | GGATATGTGGAGGGATTGAAGGATA |
| 9 | Encoding probe 9 | ATAGGAAATGGTGGTAGTGTTGGAGTTCGTTCGACTTGCATGT |
| | | ATTTCCATAGGAAATGGTGGTAGTGT |
| 10 | Encoding probe 10 | ATAGGAAATGGTGGTAGTGTGGCACCCCACCATCTAGCTAATA |
| | | CGGGCATAGGAAATGGTGGTAGTGT |
| 11 | Encoding probe 11 | ATAGGAAATGGTGGTAGTGTATTACAGAGTTTTACGAGCCGAA |
| | | ACGGAATAGGAAATGGTGGTAGTGT |
| 12 | Encoding probe 12 | ATAGGAAATGGTGGTAGTGTAAGAGTTTCCGATGCACTTCTTC |
| | | GCAAATAGGAAATGGTGGTAGTGT |
| 13 | Encoding probe 13 | ATAGGAAATGGTGGTAGTGTTTTCTCCAACACTTAGCATTCATC |
| | | GTAATATAGGAAATGGTGGTAGTGT |
| 14 | Encoding probe 14 | ATAGGAAATGGTGGTAGTGTACACCGAAGGGAACGTCTAATCT |
| | | CTATCATAGGAAATGGTGGTAGTGT |
| 15 | Encoding probe 15 | ATAGGAAATGGTGGTAGTGTAATGCTGGCAACTGATAATAAGG |
| | | GTTCGCATAGGAAATGGTGGTAGTGT |
| 16 | Encoding probe 16 | ATAGGAAATGGTGGTAGTGTCAAAGCCTACAATCCGAACTGAG |
| | | AAACCATAGGAAATGGTGGTAGTGT |
| 17 | Encoding probe 17 | AGAGTGAGTAGTAGTGGAGTGTAGTTTCCAAATGTTATCCCCC |
| | | ACTATTAGAGTGAGTAGTAGTGGAGT |
| 18 | Encoding probe 18 | AGAGTGAGTAGTAGTGGAGTCAGAATGTGGCCGATCAACCTCT |
| | | GTCAGAGTGAGTAGTAGTGGAGT |
| 19 | Encoding probe 19 | AGAGTGAGTAGTAGTGGAGTAGAAACAACAGAGTTTTACGACC |
| | | CGTTTAGAGTGAGTAGTAGTGGAGT |
| 20 | Encoding probe 20 | AGAGTGAGTAGTAGTGGAGTAAACAGTTTCCGATGCACTTCCT |
| | | CCCAAGAGTGAGTAGTAGTGGAGT |
| 21 | Encoding probe 21 | AGAGTGAGTAGTAGTGGAGTTTTCTCCAACACCTAGCATTCATC |
| | | GAAAAGAGTGAGTAGTAGTGGAGT |
| 22 | Encoding probe 22 | AGAGTGAGTAGTAGTGGAGTTGCAACCATGCACCACCTGTCAA |
| | | AAAGAGTGAGTAGTAGTGGAGT |
| 23 | Encoding probe 23 | AGAGTGAGTAGTAGTGGAGTGCCTGTCACCGGCAGTCTTACTA |
| | | GTCAAGAGTGAGTAGTAGTGGAGT |
| 24 | Encoding probe 24 | AGAGTGAGTAGTAGTGGAGTGTAGTCCCACCTTAGACGGCTCC |
| | | GAAGAGTGAGTAGTAGTGGAGT |
| 25 | Encoding probe 25 | TTGGAGGTGTAGGGAGTAAACCAGATCCCGTTCGACTTGCATG |
| | | ACATTGGAGGTGTAGGGAGTAAA |
| 26 | Encoding probe 26 | TTGGAGGTGTAGGGAGTAAAGTACATGCCACTAAACACTTTCC |
| | | CATGTTTGGAGGTGTAGGGAGTAAA |
| 27 | Encoding probe 27 | TTGGAGGTGTAGGGAGTAAATCGGCGCTTATTCGAAAGGTACA |
| | | CTGTGTTGGAGGTGTAGGGAGTAAA |
| 28 | Encoding probe 28 | TTGGAGGTGTAGGGAGTAAATAAAGTCTCCCCTACTGCACTCTT |
| | | CGTTGGAGGTGTAGGGAGTAAA |
| 29 | Encoding probe 29 | TTGGAGGTGTAGGGAGTAAAGTCTATCTAATCCTGTTCGCTCCC |
| | | CTGCTTGGAGGTGTAGGGAGTAAA |
| 30 | Encoding probe 30 | TTGGAGGTGTAGGGAGTAAAGACAACCTGCCCCGAAAGGAAA |
| | | GGTTTGGAGGTGTAGGGAGTAAA |
| 31 | Encoding probe 31 | TTGGAGGTGTAGGGAGTAAAAGCTTGCAGACTACGATCCGAAC |
| | | TCTCTTGGAGGTGTAGGGAGTAAA |
| 32 | Encoding probe 32 | TTGGAGGTGTAGGGAGTAAAAGGATCACGAGCCTCACCTTAGA |
| | | CCCGTTGGAGGTGTAGGGAGTAAA |
| 33 | Encoding probe 33 | AGGGTGTGTTTGTAAAGGGTGTTACCAAATTCATCTAGGCAAG |
| | | CTCCGAAGGGTGTGTTTGTAAAGGGT |
| 34 | Encoding probe 34 | AGGGTGTGTTTGTAAAGGGTCGTTCCATCCAAGAGTGATAGCA |
| | | GAAGGTAGGGTGTGTTTGTAAAGGGT |
| 35 | Encoding probe 35 | AGGGTGTGTTTGTAAAGGGTAAGCACTACCAACAGAGCTTTAC |
| | | GAGGGCAGGGTGTGTTTGTAAAGGGT |
| 36 | Encoding probe 36 | AGGGTGTGTTTGTAAAGGGTAGATGCACTCAAGTTCAACAGTT |
| | | TCTCTAAGGGTGTGTTTGTAAAGGGT |
| 37 | Encoding probe 37 | AGGGTGTGTTTGTAAAGGGTTACCCACCTGTCTCAGCGTCCGGC |
| | | AGGGTGTGTTTGTAAAGGGT |
| 38 | Encoding probe 38 | AGGGTGTGTTTGTAAAGGGTTGCTTTCAGAGATCCGCTTGCCTA |
| | | GCAGGGTGTGTTTGTAAAGGGT |
| 39 | Encoding probe 39 | AGGGTGTGTTTGTAAAGGGTTAGACCGGCTTTGGGTGTTACACT |
| | | GAGGGTGTGTTTGTAAAGGGT |
| 40 | Encoding probe 40 | AGGGTGTGTTTGTAAAGGGTGCTTCACCCTAATCATCTGTCCTA |
| | | CCAATAGGGTGTGTTTGTAAAGGGT |
| 41 | Encoding probe 41 | AGGTTAGGTTGAGAATAGGAAGATGGTTCAGCTCGCTCGACAA |
| | | CAGGTTAGGTTGAGAATAGGA |
| 42 | Encoding probe 42 | AGGTTAGGTTGAGAATAGGAGAAGCCGCCTTTTATAAGCTGAT |
| | | CAACGAGGTTAGGTTGAGAATAGGA |
| 43 | Encoding probe 43 | AGGTTAGGTTGAGAATAGGACATTCTACGGATTGCACTAGATG |
| | | TCATCTAGGTTAGGTTGAGAATAGGA |
| 44 | Encoding probe 44 | AGGTTAGGTTGAGAATAGGACCGGCTCCTCGTTGTACTGTCCA |
| | | AACAGGTTAGGTTGAGAATAGGA |
| 45 | Encoding probe 45 | AGGTTAGGTTGAGAATAGGAGTACCGAAGTGAATCTGTTGGTT |
| | | CACGAAGGTTAGGTTGAGAATAGGA |
| 46 | Encoding probe 46 | AGGTTAGGTTGAGAATAGGACTGCTTACGCCGCCTTTTATAAG |
| | | CTCTAAGGTTAGGTTGAGAATAGGA |
| 47 | Encoding probe 47 | AGGTTAGGTTGAGAATAGGAAGAGGATTGCACTAGATGTCAAG |
| | | ACCACCAGGTTAGGTTGAGAATAGGA |
| 48 | Encoding probe 48 | AGGTTAGGTTGAGAATAGGATCCTGCTCCTCGTTGTACTGTCCT |
| | | AAAGGTTAGGTTGAGAATAGGA |
| 49 | Encoding probe 49 | GATGATGTAGTAGTAAGGGTGCGGCTTTCCTAACGTCATTACC |
| | | GAACATGATGATGTAGTAGTAAGGGT |
| 50 | Encoding probe 50 | GATGATGTAGTAGTAAGGGTACCAGCCTTTACCTTACCAACTA |
| | | GCTTTAGATGATGTAGTAGTAAGGGT |
| 51 | Encoding probe 51 | GATGATGTAGTAGTAAGGGTATGATGGCACTAGATGTCAAGAC |
| | | CTCCAGATGATGTAGTAGTAAGGGT |
| 52 | Encoding probe 52 | GATGATGTAGTAGTAAGGGTTTGAGAACAGCTTTCAGAGATTC |
| | | GCAACGATGATGTAGTAGTAAGGGT |
| 53 | Encoding probe 53 | GATGATGTAGTAGTAAGGGTTCAATCTGTTAGTTCCGCTCGCTG |
| | | CTGATGATGTAGTAGTAAGGGT |
| 54 | Encoding probe 54 | GATGATGTAGTAGTAAGGGTAACAAGCCTTTACCTTACCAACT |
| | | AGCATTGATGATGTAGTAGTAAGGGT |
| 55 | Encoding probe 55 | GATGATGTAGTAGTAAGGGTACGCACCTGTCTTAGCGTCCCCCT |
| | | TGATGATGTAGTAGTAAGGGT |
| 56 | Encoding probe 56 | GATGATGTAGTAGTAAGGGTTGAGAACAGCTTTCAGAGATTCG |
| | | CTACGGATGATGTAGTAGTAAGGGT |
| 57 | Encoding probe 57 | TAGAGTTGATAGAGGGAGAAGCTGCCTCCCGTAGGAGT |
| 58 | Readout probe 1 | /5Alex488N/TATCCTTCAATCCCTCCACA |
| 59 | Readout probe 2 | /5Alcx546N/ACACTACCACCATTTCCTAT |
| 60 | Readout probe 3 | /56-ROXN/ACTCCACTACTACTCACTCT/3Rox_N/ |
| 61 | Readout probe 4 | /5Atto610N/TTTACTCCCTACACCTCCAA |
| 62 | Readout probe 5 | /5Alex647N/ACCCTTTACAAACACACCCT |
| 63 | Readout probe 6 | /5DyLight-510-LS/TCCTATTCTCAACCTAACCT/3DyLight-510-LS/ |
| 64 | Readout probe 7 | /5Alex405N/TTCTCCCTCTATCAACTCTA |
| 65 | Readout probe 8 | /5Alex532N/ACCCTTACTACTACATCATC/3Alexa532N/ |

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1. A screening method, comprising:
(i) providing:
(a) a sample;
(b) a pathogen;
(c) at least one microbial strain, and
(d) at least one set of probes, wherein the at least one set of probes comprises at least one encoding probe and at least one emissive readout probe;
(ii) performing an imaging assay, wherein the imagining assay comprises:
(A) contacting the (a) sample with the (b) pathogen and the (c) at least one microbial strain to produce a first complex;
(B) contacting the at least one encoding probe with the first complex to form a second complex, wherein the at least one encoding probe comprises a targeting sequence and a landing pad sequence,
(C) contacting the at least one emissive readout probe with the second complex, wherein the at least one emissive readout probe comprises a label and a sequence complementary to the landing pad sequence;
(D) acquiring one or more emission spectra from the at least one emissive readout probe;
(E) repeating the aforementioned steps for at least one different encoding probe;
(F.1) determining the spectra of “signal” and assigning them to a species of the at least one microbial strain;
(F.2) determining the spectra of “signal” and assigning them to a species of the pathogen;
(G) decoding the spectra into a single, targeted transcript through means of signal deconvolution, error correction, comparison to reference standards; and
(iii) quantifying the association between the at least one microbial strain and the sample as compared to the association between the pathogen and the sample, thereby determining the therapeutic potential of the at least one microbial strain to treat or prevent a disease caused by the pathogen.

2. A screening method comprising:
(i) providing:
(a) a sample;
(b) a pathogen;
(c) at least one microbial strain, and
(d) at least one set of probes, wherein the at least one set of probes comprises at least one encoding probe and at least one emissive readout probe;
(ii) performing an imaging assay, wherein the imagining assay comprises:
(A) contacting the (a) sample with the (b) pathogen and the (c) at least one microbial strain to produce a first complex;
(B) contacting the at least one encoding probe with the first complex to form a second complex, wherein the at least one encoding probe comprises a targeting sequence and a landing pad sequence,
(C) contacting the at least one emissive readout probe with the second complex, wherein the at least one emissive readout probe comprises a label and a sequence complementary to the landing pad sequence;
(D) acquiring one or more emission spectra from the at least one emissive readout probe;
(E) repeating the aforementioned steps for at least one different encoding probe;
(F.1) determining the spectra of “signal” and assigning them to a species of the at least one microbial strain;
(F.2) determining the spectra of “signal” and assigning them to a species of the pathogen;
(G) decoding the spectra into a single, targeted transcript through means of signal deconvolution, error correction, comparison to reference standards; and
(iii) quantifying the association between the at least one microbial strain and the sample as compared to the association between the pathogen and the sample, thereby determining the potential of the at least one microbial strain to be a live biotherapeutic product (LBP).

3. The method of claim 1, wherein the sample comprises at least one cell, a cell suspension, a tissue biopsy, a tissue specimen, bone biopsies, organoids, three-dimensional hydrogel scaffolds, transwell systems, or plant biopsies.

4. The method of claim 1, wherein the sample is a cell; optionally, wherein the cell is a bacterial or eukaryotic cell.

5. The method of claim 1, wherein the pathogen is a disease-causing microorganism selected from the group consisting of a bacterium, a fungus, a virus, an archaea, a phage, a prion, and a parasite, or a fragment thereof.

6. The method of claim 5, wherein the pathogen is a disease-causing microorganism that causes a urogenital (e.g., bacterial vaginosis, vulvovaginal candidiasis, sexually transmitted diseases, urinary tract infection), skin (e.g., atopic dermatitis, acne, methicillin-resistant Straphylococcus aureus (MRSA), epidermolysis bullosa), lung (e.g., cystic fibrosis, Mycobacterium tuberculosis, pneumonia), or gastrointestinal (e.g., stomach ulcer, ulcerative colitis, Chron's disease, irritable bowel syndrome, C. difficile infection, celiac's disease, lactose intolerance, food allergies, small intestine bacterial overgrowth, viral gastroenteritis, necrotizing enterocolitis, gingivitis) disease.

7. The method of claim 1, wherein the at least one microbial strain is a strain derived from a microbe selected from the group consisting of a bacterium, a synthetic bacterium, a synthetic organism, a fungus, a virus, an archaea, a parasite, and a genetically modified organism; optionally, wherein the at least one microbial strain is a bacterial strain.

8. The method of claim 1, wherein the at least one microbial strain is a genetically engineered strain; optionally, wherein the at least one microbial strain is a genetically engineered microbial strain.

9. The method of claim 1, wherein the targeting sequence targets at least one of messenger RNA (mRNA), micro RNA (miRNA), long non-coding RNA (lncRNA), ribosomal RNA (rRNA), small interfering RNA (siRNA), transfer RNA (tRNA), Crispr RNA (crRNA), trans-activating cirspr RNA (tracrRNA), mitochondria RNA, Intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigenic target.

10. The method of claim 9, wherein the targeting sequence targets mRNA and/or rRNA.

11. The method of claim 1, wherein
(a) the targeting sequence targets a 16S rRNA sequence, a 5S rRNA sequence, and/or a 23S rRNA sequence in the microbial strain; and/or
(b) the targeting sequence targets a 16S rRNA sequence, a 5S rRNA sequence, and/or a 23S rRNA sequence in the pathogen.

12. The method of claim 1, wherein the targeting sequence targets a 18S rRNA sequence, a 5.8S rRNA sequence, and/or a 28S rRNA sequence in the in the microbial strain, wherein the microbial strain is a eukaryotic microbial strain.

13. The method of claim 1, wherein the emissive readout probe comprises a label on the 5′ and/or 3′ end.

14. The method of claim 13, wherein the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

15. The method of claim 13, wherein the label is imaged using widefield epifluorescence microscopy, widefield phase contrast microscopy, point scanning confocal microscopy, spinning disk confocal microscopy, lattice lightsheet microscopy, or light field microscopy.

16. The method of claim 15, wherein the detection strategy used is channel, spectral, channel and fluorescence lifetime, or spectral and fluorescence lifetime.

17. The method of claim 1, wherein the sample is on an analyzing platform, wherein the analyzing platform is a microscope slide, at least one chamber, at least one microfluidic device, at least one well, at least one plate, or at least one filter membrane.

18. A library of constructs, each construct comprising:
(a) a target cell;
(b) a first plurality of encoding and readout probes, wherein each encoding probe comprises a targeting sequence that is a region of interest on a nucleotide of a pathogen and a landing pad sequence; and each readout probe comprises a label and a sequence complementary to the landing pad sequence; and
(c) a second plurality of encoding and readout probes, wherein each encoding probe comprises a targeting sequence that is a region of interest on a nucleotide of a microbial strain and a landing pad sequence; and each readout probe comprises a label and a sequence complementary to the landing pad sequence.

19. A library of constructs, each construct comprising:
(a) a target cell;
(b) a first plurality of encoding and readout probes, wherein each encoding probe comprises a targeting sequence that is a region of interest on a nucleotide of a pathogen and a landing pad sequence; and each readout probe comprises a label and a sequence complementary to the landing pad sequence; and
(c) a second plurality of encoding and readout probes, wherein each encoding probe comprises a targeting sequence that is a region of interest on a nucleotide of a bacterial strain and a landing pad sequence; and each readout probe comprises a label and a sequence complementary to the landing pad sequence.

20. A method for determining the spatial location of a complex, comprising:
(i) providing:
(a) a target cell;
(b) a pathogen;
(c) at least one microbial strain, and
(d) at least one set of probes, wherein the at least one set of probes comprises at least one encoding probe and at least one emissive readout probe;
(ii) performing an imaging assay, wherein the imagining assay comprises:
(A) contacting the (a) target cell with the (b) pathogen and the (c) at least one microbial strain to produce a first complex;
(B) contacting the at least one encoding probe with the first complex to form a second complex, wherein the at least one encoding probe comprises a targeting sequence and a landing pad sequence,
(C) contacting the at least one emissive readout probe with the second complex, wherein the at least one emissive readout probe comprises a label and a sequence complementary to the landing pad sequence;
(D) acquiring one or more emission spectra from the at least one emissive readout probe;
(E) repeating the aforementioned steps for at least one different encoding probe;
(F.1) determining the spectra of “signal” and assigning them to a species of the at least one microbial strain;
(F.2) determining the spectra of “signal” and assigning them to a species of the pathogen;
(G) decoding the spectra into a single, targeted transcript through means of signal deconvolution, error correction, comparison to reference standards;
(H) performing a second assay to image the target cell; and
(iii) quantify the abundance of the at least one microbial strain to the target cell and/or the association between the pathogen to the target cell, thereby determining the spatial location of the complex,
wherein the complex is a target cell-microbial strain complex or a target cell-pathogen complex.

21. A method for determining the spatial location of a complex, comprising:
(i) providing:
(a) a target cell;
(b) a pathogen;
(c) at least one bacterial strain, and
(d) at least one set of probes, wherein the at least one set of probes comprises at least one encoding probe and at least one emissive readout probe;
(ii) performing an imaging assay, wherein the imagining assay comprises:
(A) contacting the (a) target cell with the (b) pathogen and the (c) at least one bacterial strain to produce a first complex;
(B) contacting the at least one encoding probe with the first complex to form a second complex, wherein the at least one encoding probe comprises a targeting sequence and a landing pad sequence,
(C) contacting the at least one emissive readout probe with the second complex, wherein the at least one emissive readout probe comprises a label and a sequence complementary to the landing pad sequence;
(D) acquiring one or more emission spectra from the at least one emissive readout probe;
(E) repeating the aforementioned steps for at least one different encoding probe;
(F.1) determining the spectra of “signal” and assigning them to a species of the at least one bacterial strain;
(F.2) determining the spectra of “signal” and assigning them to a species of the pathogen;
(G) decoding the spectra into a single, targeted transcript through means of signal deconvolution, error correction, comparison to reference standards;
(H) performing a second assay to image the target cell; and
(iii) quantify the abundance of the at least one bacterial strain to the target cell and/or the association between the pathogen to the target cell, thereby determining the spatial location of the complex,
wherein the complex is a target cell-bacterial strain complex or a target cell-pathogen complex.
